(19) United States
(12) Patent Application Publication
(10) Pub. No.: US 2019/0282210 A1
(43) Pub. Date: Sep. 19, 2019

(54) ULTRASOUND OBSERVATION DEVICE, AND METHOD FOR OPERATING ULTRASOUND OBSERVATION DEVICE

(71) Applicant: OLYMPUS CORPORATION, Tokyo (JP)

(72) Inventor: Tomohiro NAKATSUJI, Hamburg (DE)

(73) Assignee: OLYMPUS CORPORATION, Tokyo (JP)

(21) Appl. No.: 16/427,641

(22) Filed: May 31, 2019

Related U.S. Application Data
(63) Continuation of application No. PCT/JP2017/044445, filed on Dec. 11, 2017.

(30) Foreign Application Priority Data
Dec. 19, 2016 (JP) 2016-245746

(51) Int. Cl. A61B 8/08; A61B 8/00

(52) U.S. Cl. CPC A61B 8/5207; A61B 8/5269; A61B 8/14; A61B 8/469; A61B 8/08

(57) ABSTRACT

An ultrasound observation device includes a processor configured to execute: generating an ultrasound image; setting at least two regions of interest on the ultrasound image; calculating feature value on each of the set regions based on the ultrasound signal; calculating a representative value on each of the set regions based on the calculated feature value of each of the set regions; selecting at least one representative value from the representatives values of the set regions; selecting the feature value having a predetermined relationship with the selected representatives value from the feature value used for calculating the selected representatives value; setting the selected feature value as a threshold; setting, as a display specification, a color pattern of the feature value to be displayed on a display based on the set threshold; and generating feature-value image data in which the feature value is colored with the set display specification.

This application is a continuation of PCT International Application No. PCT/JP2017/044445 filed on Dec. 11, 2017, which designates the United States, incorporated herein by reference, and which claims the benefit of priority from Japanese Patent Application No. 2016-245746, filed on Dec. 19, 2016, incorporated herein by reference.

### BACKGROUND

The present disclosure relates to an ultrasound observation device, and a method for operating the ultrasound observation device.

Ultrasound waves are sometimes used to observe the characteristics of living tissue or material that is the observation target. Specifically, ultrasound waves are transmitted to the observation target, and predetermined signal processing is executed on ultrasound echoes reflected by the observation target so that information about the characteristics of the observation target is acquired.

Specifically, as a technique for observing tissue characteristics of the observation target, such as subject, by using ultrasound waves, there is a known technique for obtaining, as an image, the feature value on a frequency spectrum of a received ultrasound signal (for example, see Japanese Patent No. 5303147). According to this technique, after displacement data is calculated at each measurement point from data on multiple frames as the value representing the tissue characteristics of the observation target, the degree of elasticity is obtained as a feature value from the displacement data, and an elasticity image with the visual information corresponding to the feature value assigned thereto is generated and displayed. A user such as a doctor views the displayed elasticity image to diagnose tissue characteristics of the subject.

For example, according to Japanese Patent No. 5303147, a single region of interest is set and, as the elasticity image that is obtained as an image based on the hardness of the target tissue to be observed in the region of interest, the image in which a color is assigned corresponding to the feature value is displayed. The elasticity image is typically called elastography; information about the hardness (the degree of elasticity) of the observation target in the set region is acquired, and color information corresponding to a feature value is superimposed on the ultrasound image. Specifically, in Japanese Patent No. 5303147, based on the upper limit value and the lower limit value that are previously set, a color phase code obtained for gradation is assigned to a measurement point of the measurement target. This makes it possible to display, on the display device, an elasticity image in which a color phase is changed in accordance with the degree of elasticity.

### SUMMARY

An ultrasound observation device according to one aspect of the present disclosure includes a processor, the processor being configured to execute: generating an ultrasound image based on an ultrasound signal acquired by an ultrasound probe including an ultrasound transducer configured to transmit an ultrasound wave to an observation target and receive an ultrasound wave reflected by the observation target; setting at least two regions of interest on the ultrasound image; calculating a feature value on each of the set regions of interest based on the ultrasound signal; calculating a representative value on each of the set regions of interest based on the calculated feature value of each of the set regions of interest; selecting at least one representative value from the representatives values of the set regions of interest; selecting the feature value having a predetermined relationship with the selected representatives value from the feature value used for calculating the selected representatives value; setting the selected feature value as a threshold; setting, as a display specification, a color pattern of the feature value to be displayed on a display based on the set threshold; and generating feature-value image data in which the feature value, displayed together with the ultrasound image, is colored with the set display specification.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram that illustrates a configuration of an ultrasound observation system including an ultrasound observation device according to a first embodiment;

FIG. 2 is a graph that illustrates the relationship between a receive depth and an amplification factor during an amplification process performed by a signal amplifying unit in the ultrasound observation device according to the first embodiment;

FIG. 3 is a graph that illustrates the relationship between the receive depth and the amplification factor during an amplification correction process performed by an amplification correcting unit in the ultrasound observation device according to the first embodiment;

FIG. 4 is a diagram that schematically illustrates the data arrangement in a single sound ray of ultrasound signals;

FIG. 5 is a graph that illustrates an example of the frequency spectrum calculated by a frequency analyzing unit in the ultrasound observation device according to the first embodiment;

FIG. 6 is a graph that illustrates a straight line having, as parameters, the feature value corrected by the attenuation correcting unit in the ultrasound observation device according to the first embodiment;

FIG. 7 is a diagram that illustrates a process performed by a display-specification setting unit in the ultrasound observation device according to the first embodiment;

FIG. 8 is a flowchart that illustrates the outline of a process performed by the ultrasound observation device according to the first embodiment;

FIG. 9 is a flowchart that illustrates the outline of the process performed by the frequency analyzing unit in the ultrasound observation device according to the first embodiment;

FIG. 10 is a diagram that schematically illustrates a display example of the feature-value image on the display device in the ultrasound observation device according to the first embodiment;

FIG. 11 is a diagram that illustrates a process performed by a display-specification setting unit in the ultrasound observation device according to a modification 1 of the first embodiment;

FIG. 12 is a diagram that schematically illustrates an example of the display on the display device in the ultrasound observation device according to a modification 2 of the first embodiment;

FIG. 13 is a diagram that illustrates a process performed by a display-specification setting unit in the ultrasound observation device according to a second embodiment;

FIG. 14 is a block diagram that illustrates a configuration of an ultrasound observation system including an ultrasound observation device according to a third embodiment;

FIG. 15 is a diagram that illustrates a process performed by the display-specification setting unit in the ultrasound observation device according to the third embodiment;

FIG. 16 is a block diagram that illustrates a configuration of an ultrasound observation system including an ultrasound observation device according to a fourth embodiment;

FIG. 17 is a block diagram that illustrates a configuration of an ultrasound observation system including an ultrasound observation device according to a fifth embodiment; and

FIG. 18 is a flowchart that illustrates the outline of a process performed by the ultrasound observation device according to the fifth embodiment.

### DETAILED DESCRIPTION

With reference to the accompanying drawings, an aspect (hereinafter, referred to as “embodiment”) for carrying out the present disclosure is explained below.

### First Embodiment

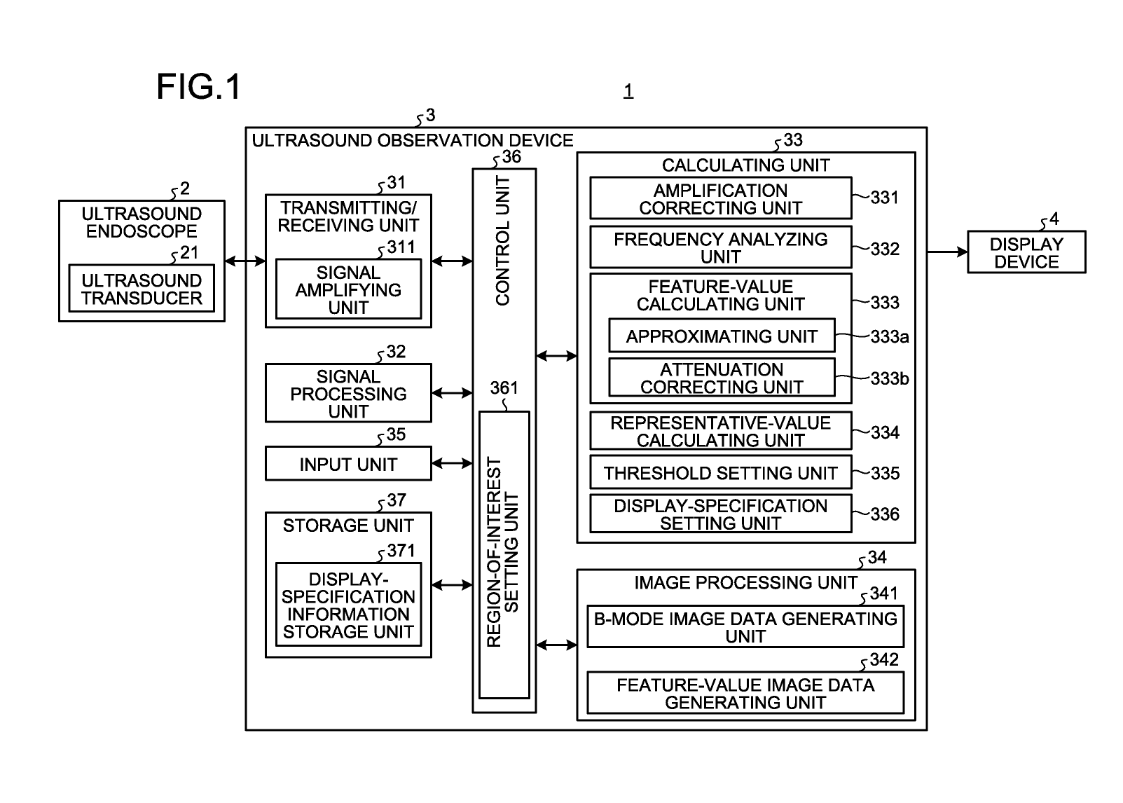

FIG. 1 is a block diagram that illustrates a configuration of an ultrasound observation system 1 including an ultrasound observation device 3 according to a first embodiment. The ultrasound observation system 1 illustrated in the drawing includes: an ultrasound endoscope 2 (ultrasound probe) that transmits ultrasound waves to a subject, which is the observation target, and receives ultrasound waves reflected by the subject; the ultrasound observation device 3 that generates ultrasound images based on ultrasound signals acquired by the ultrasound endoscope 2; and a display device 4 that displays ultrasound images generated by the ultrasound observation device 3.

At the distal end of the ultrasound endoscope 2, an ultrasound transducer 21 is provided which converts electric pulse signals received from the ultrasound observation device 3 into ultrasound pulses (sound pulses) and emits them to the subject and also converts ultrasound echoes reflected by the subject into electric echo signals represented by changes in a voltage and outputs them. The ultrasound transducer 21 may be any of a convex transducer, a linear transducer, and a radial transducer. The ultrasound endoscope 2 may cause the ultrasound transducer 21 to conduct scanning mechanically or may cause it to conduct scanning electronically with elements arranged in array as the ultrasound transducer 21 by electronically switching elements for transmitting/receiving or by applying a delay for each element in transmitting/receiving.

The ultrasound endoscope 2 typically includes an optical imaging system and an imaging element, and it is inserted into a digestive tract (esophagus, stomach, duodenum, large intestine) or respiratory apparatus (trachea, bronchi) of the subject so as to capture the digestive tract, respiratory apparatus, or their periphery organs (pancreas, gallbladder, bile duct, biliary tract, lymph node, mediastinal organ, blood vessel, or the like). Furthermore, the ultrasound endoscope 2 includes a light guide that guides illumination light emitted to the subject during capturing. The distal end of the light guide reaches the distal end of the insertion unit of the ultrasound endoscope 2 for the subject while the proximal end thereof is connected to a light source device that generates the illumination light. Moreover, not only the ultrasound endoscope 2 but also an ultrasound probe that does not including an optical imaging system or an imaging element may be used.

The ultrasound observation device 3 includes: a transmitting/receiving unit 31 that is electrically connected to the ultrasound endoscope 2 so that it transmits transmission signals (pulse signals) that are high-voltage pulses based on a predetermined waveform and transmission timing to the ultrasound transducer 21 and receives echo signals, which are electric receive signals, from the ultrasound transducer 21 to generate and output digital high-frequency (RF: Radio Frequency) signal data (hereafter, referred to as RF data); a signal processing unit 32 that generates digital B-mode receive data based on RF data received from the transmitting/receiving unit 31; a calculating unit 33 that performs predetermined calculations on RF data received from the transmitting/receiving unit 31; an image processing unit 34 that generates various types of image data; an input unit 35 that is implemented by using a user interface, such as keyboard, mouse, or touch panel, and receives inputs of various types of information; a control unit 36 that controls the overall ultrasound observation system 1; and a storage unit 37 that stores various types of information needed for operation of the ultrasound observation device 3.

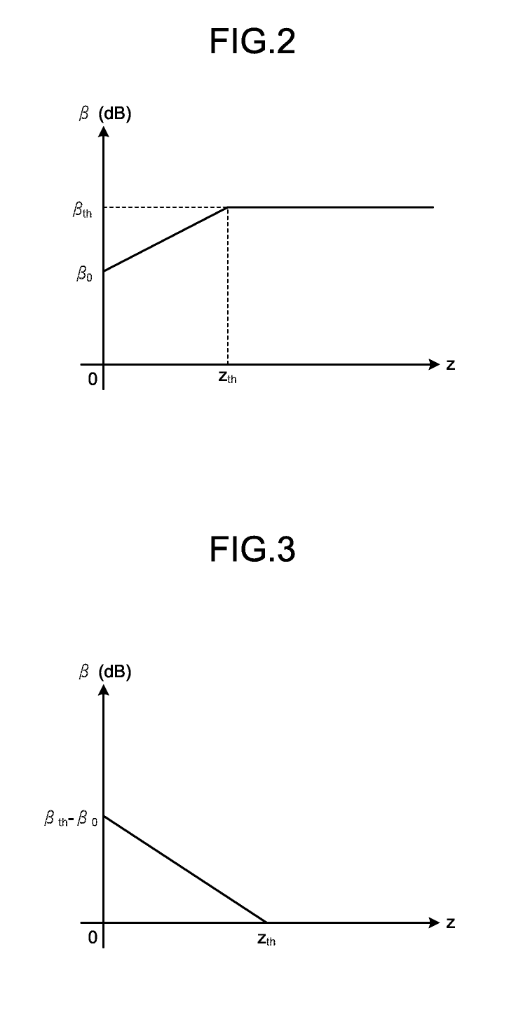

The transmitting/receiving unit 31 includes a signal amplifying unit 311 that amplifies echo signals. The signal amplifying unit 311 conducts STC (Sensitivity Time Control) correction to amplify an echo signal having a larger receive depth with a higher amplification factor. FIG. 2 is a graph that illustrates the relationship between a receive depth and an amplification factor during an amplification process performed by the signal amplifying unit 311. A receive depth z illustrated in FIG. 2 is a value calculated based on the time elapsing from the time when reception of an ultrasound wave is started. As illustrated in FIG. 2, an amplification factor β(dB) linearly increases from β₀ to βₜₕ (>β₀) in accordance with an increase in the receive depth z when the receive depth z is smaller than the threshold zₜₕ. Furthermore, the amplification factor β has a constant value βₜₕ when the receive depth z is equal to or more than the threshold zₜₕ. The value of the threshold zₜₕ is a value with which most of the ultrasound signal received from the observation target has been attenuated and noises are dominant. More generally, the amplification factor β may monotonically increase in accordance with an increase in the receive depth z when the receive depth z is smaller than the threshold zₜₕ. Here, the relationship illustrated in FIG. 2 is previously stored in the storage unit 37.

After performing processing such as filtering on an echo signal amplified by the signal amplifying unit 311, the transmitting/receiving unit 31 conducts A/D conversion to generate time-domain RF data and outputs it to the signal processing unit 32 and the calculating unit 33. Furthermore, when the ultrasound endoscope 2 has a configuration such that the ultrasound transducer 21 having a plurality of elements arranged in array is caused to conduct electronic scanning, the transmitting/receiving unit 31 includes a multi-channel circuit for beam synthesis that corresponds to the elements.

The frequency band of pulse signals transmitted by the transmitting/receiving unit 31 may be a wide band that almost covers the linear-response frequency band for electroacoustic conversion from pulse signals into ultrasound pulses by the ultrasound transducer 21. Furthermore, the frequency band for various types of processing on echo signals in the signal amplifying unit 311 may be a wide band that almost covers the linear-response frequency band for electroacoustic conversion from ultrasound echoes into echo signals by the ultrasound transducer 21. This allows high-accuracy approximation when an approximation process is performed on a frequency spectrum described later.

The transmitting/receiving unit 31 has functions to transmit various control signals output from the control unit 36 to the ultrasound endoscope 2 and also receive various types of information including ID for identification from the ultrasound endoscope 2 and transmits it to the control unit 36.

The signal processing unit 32 performs known processing such as bandpass filtering, envelope detection, or logarithmic conversion on RF data to generate digital B-mode receive data. For logarithmic conversion, the common logarithm of the amount of division of RF data by the reference voltage V_{c} is represented by a decibel value. The signal processing unit 32 outputs the generated B-mode receive data to the image processing unit 34. The signal processing unit 32 is implemented by using a CPU (Central Processing Unit), various types of arithmetic circuits, or the like.

The calculating unit 33 includes: an amplification correcting unit 331 that conducts amplification correction on RF data generated by the transmitting/receiving unit 31 such that the amplification factor β is constant regardless of the receive depth z; a frequency analyzing unit 332 that executes frequency analysis by conducting fast Fourier transform (FFT) on RF data, on which the amplification correction has been performed, to calculate a frequency spectrum; a feature-value calculating unit 333 that calculates a feature value on a frequency spectrum, calculated by the frequency analyzing unit 332, based on the frequency spectrum; a representative-value calculating unit 334 that calculates a representative value of the feature value, which is the target to be displayed, based on the feature value calculated by the feature-value calculating unit 333; a threshold setting unit 335 that sets a threshold based on the representative value calculated by the representative-value calculating unit 334; and a display-specification setting unit 336 that sets the display specification for the feature value, which is the target to be displayed on the display device 4, based on the threshold set by the threshold setting unit 335. The calculating unit 33 is implemented by using a CPU, various types of arithmetic circuits, or the like.

FIG. 3 is a graph that illustrates the relationship between the receive depth and the amplification factor during an amplification correction process performed by the amplification correcting unit 331. As illustrated in FIG. 3, the amplification factor β(dB) during the amplification correction process performed by the amplification correcting unit 331 has the maximal value βₜₕ−β₀ when the receive depth z is zero, it linearly decreases when the receive depth z reaches the threshold zₜₕ from zero, and it is zero when the receive depth z is equal to or more than the threshold zₜₕ. The amplification correcting unit 331 amplifies and corrects a digital RF signal with the amplification factor defined as described above, thereby canceling out effects of the STC correction performed by the signal processing unit 32 and outputting signals with the constant amplification factor βₜₕ. Furthermore, it is obvious that the relationship between the receive depth z and the amplification factor β, conducted by the amplification correcting unit 331, is different depending on the relationship between the receive depth and the amplification factor in the signal processing unit 32.

The reason why the above amplification correction is conducted is explained. The STC correction is a correction process to remove effects of attenuation from the amplitude of an analog signal waveform by uniformly amplifying the amplitude of the analog signal waveform over the entire frequency band and at the amplification factor that monotonically increases with respect to the depth. Therefore, when a B-mode image is generated, which is displayed by converting the amplitude of an echo signal into luminance, and when uniform tissue is scanned, the STC correction is conducted so that a luminance value is constant regardless of a depth. That is, it is possible to obtain an advantage such that the effects of attenuation are removed from luminance values of a B-mode image.

However, in the case of use of a result of calculation and analysis of the frequency spectrum of an ultrasound wave as in the present embodiment, the STC correction does not accurately remove the effects of attenuation due to propagation of the ultrasound wave. This is because, although the attenuation is typically different depending on a frequency (see Equation (1) described later), the amplification factor of the STC correction changes depending on only the distance and it does not have frequency dependency.

To solve the above-described problem, i.e., the problem in that, when the result of calculation and analysis on the frequency spectrum of an ultrasound wave is used, the STC correction does not accurately remove effects of attenuation due to the propagation of the ultrasound wave, it is possible that a receive signal, on which STC correction has been performed, is output when a B-mode image is generated, while new transmission different from the transmission for generating a B-mode image is conducted when an image is generated based on the frequency spectrum so that a receive signal on which the STC correction has not been performed is output. In this case, however, there is a problem of a reduction in the frame rate of image data generated based on a receive signal.

Therefore, according to the present embodiment, while the frame rate of generated image data is maintained, the amplification correcting unit 331 corrects the amplification factor to remove effects of the STC correction on the signal on which the STC correction has been performed for a B-mode image.

The frequency analyzing unit 332 samples RF data (line data) on each sound ray, amplified and corrected by the amplification correcting unit 331, at a predetermined time interval and generates sample data. The frequency analyzing unit 332 performs FFT processing on a sample data group, thereby calculating a frequency spectrum at multiple points (data positions) of the RF data. The “frequency spectrum” mentioned here means “the frequency distribution of the intensity in the certain receive depth z” which is obtained when FFT processing is performed on a sample data group. Furthermore, the “intensity” mentioned here refers to, for example, any of a parameter such as the voltage of an echo signal, the power of an echo signal, the sound pressure of an ultrasound echo, or the sound energy of an ultrasound echo, the amplitude or the time integral value of the parameters, and the combination thereof.

Generally, when the observation target is living tissue, a frequency spectrum exhibits a different tendency depending on the characterization of the living tissue scanned with ultrasound waves. This is because a frequency spectrum is correlated to the size of a scattering substance that scatters ultrasound waves, the number density, the acoustic impedance, or the like. The “characterization of living tissue” mentioned here refers to, for example, malignant tumor (cancer), benign tumor, endocrine tumor, mucinous tumor, normal tissue, cyst, or vascular channel.

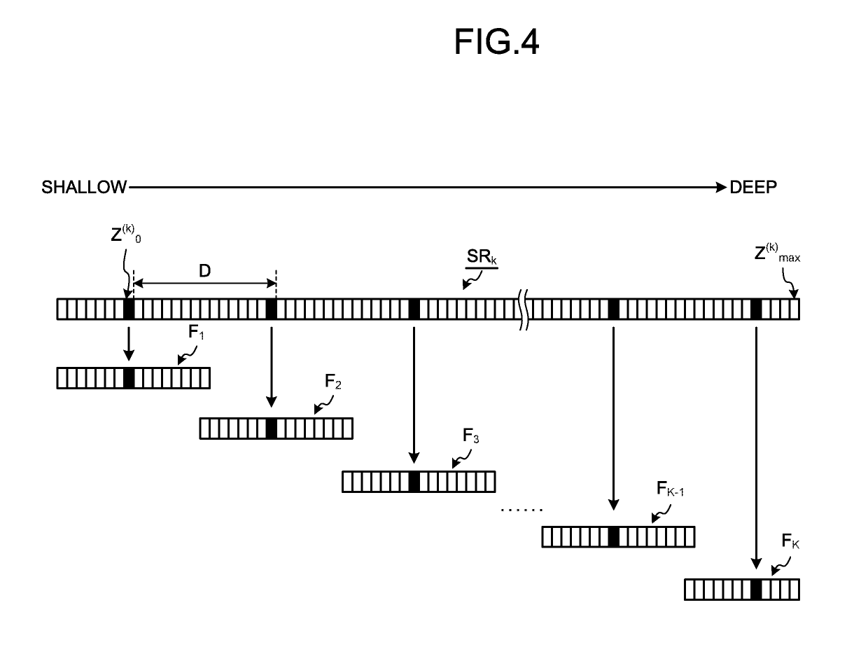

FIG. 4 is a diagram that schematically illustrates the data arrangement in a single sound ray of ultrasound signals. In a sound ray SRₖ illustrated in the drawing, a white or black rectangle represents data at a single sample point. Furthermore, in the sound ray SRₖ, data positioned on the right side is sample data at a deep position when measured along the sound ray SRₖ from the ultrasound transducer 21 (see the arrow in FIG. 4). The sound ray SRₖ is discretized at a time interval that corresponds to the sampling frequency (e.g., 50 MHz) for the A/D conversion conducted by the transmitting/receiving unit 31. In the case illustrated in FIG. 4, the position of the eighth data in the sound ray SRₖ with the number k is set as the initial value Z^{(k)}₀ in the direction of the receive depth z, the position of the initial value may be optionally set. A calculation result by the frequency analyzing unit 332 is obtained as a complex number, and it is stored in the storage unit 37.

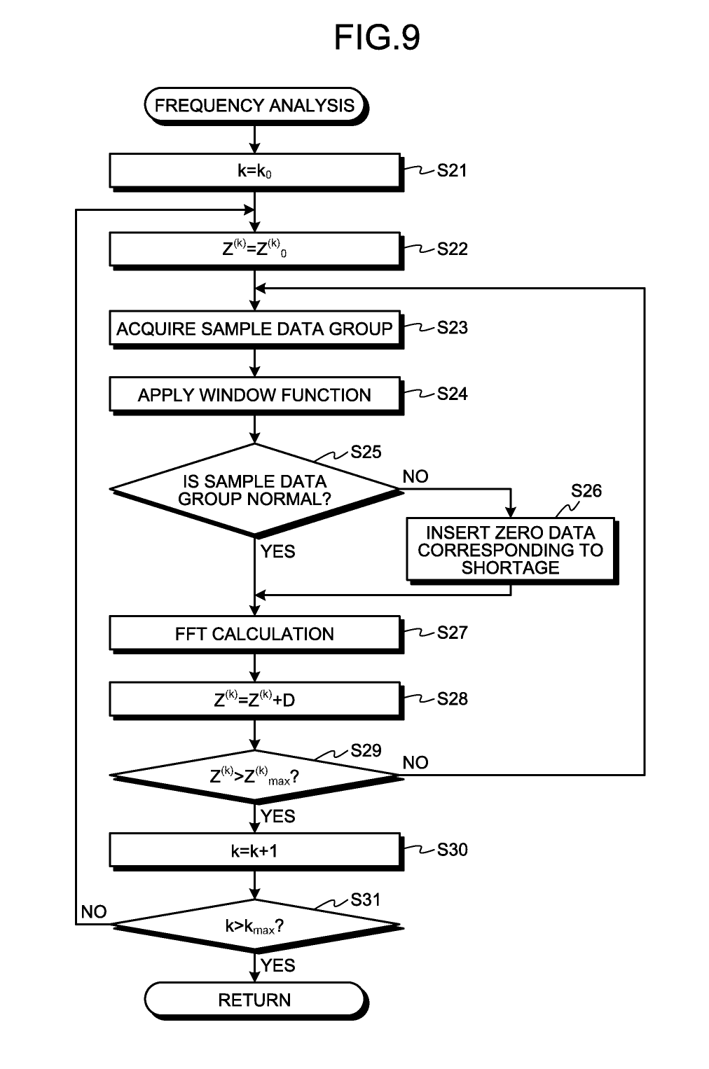

The data group Fⱼ (j=1, 2, . . . , K) illustrated in FIG. 4 is a sample data group targeted for FFT processing. Typically, to conduct FFT processing, the sample data group needs to have the number of sets of data that is a power of two. In this meaning, the sample data group Fⱼ (j=1, 2, . . . , K−1) with the number of sets of data of 16 (=2⁴) is a normal data group, meanwhile a sample data group F_{K} with the number of sets of data of 12 is a faulty data group. To conduct FFT processing on a faulty data group, a process is performed to insert zero data corresponding to a shortage so as to generate a normal sample data group. This aspect is explained in detail when a process of the frequency analyzing unit 332 is explained (see FIG. 9).

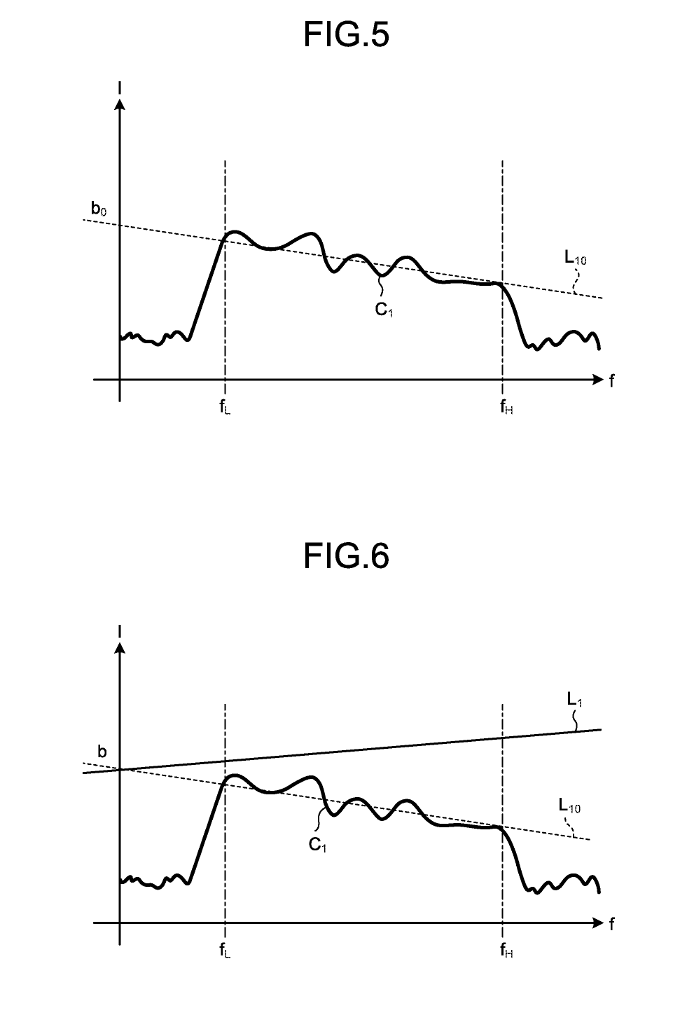

FIG. 5 is a graph that illustrates an example of the frequency spectrum calculated by the frequency analyzing unit 332. In FIG. 5, the horizontal axis is a frequency f. Furthermore, in FIG. 5, the vertical axis is I=10 log₁₀(I₀/I_{c}), which is the common logarithm (decibel representation) of the value obtained when the intensity I₀ is divided by the reference intensity I_{c} (constant). A straight line L₁₀ illustrated in FIG. 5 is described later. Furthermore, according to the present embodiment, a curved line and a straight line are composed of a set of discrete points.

As for a frequency spectrum C₁ illustrated in FIG. 5, a lower-limit frequency f_{L} and an upper-limit frequency f_{H} in the frequency band used for subsequent calculations are parameters that are determined based on the frequency band of the ultrasound transducer 21, the frequency band of pulse signals transmitted by the transmitting/receiving unit 31, or the like. Hereafter, in FIG. 5, the frequency band defined by the lower-limit frequency f_{L} and the upper-limit frequency f_{H} is referred to as “frequency band F”.

The feature-value calculating unit 333 calculates the feature value on each of frequency spectra in the set region of interest (hereafter, sometimes referred to as ROI (Region of Interest)). In the first embodiment, an explanation is given based on the assumption that two regions of interest having regions different from each other are set. The feature-value calculating unit 333 includes: an approximating unit 333a that approximates a frequency spectrum with a straight line, thereby calculating a feature value (hereafter, referred to as pre-correction feature value) on the frequency spectrum on which an attenuation correction process has not been performed; and an attenuation correcting unit 333b that conducts attenuation correction on the pre-correction feature value, calculated by the approximating unit 333a, thereby calculating a feature value.

Furthermore, a spatial filter such as a smoothing filter may be applied to data used by the feature-value calculating unit 333 to calculate a feature value. Here, an indicator as to whether a spatial filter is used or not may be used. For example, “ON” is displayed in green when a spatial filter is used, and “OFF” is displayed in white when no spatial filter is used. For example, “ON” or “OFF” is displayed just under the attenuation correction indication (the area displaying information such as an attenuation rate).

The approximating unit 333a executes regression analysis on a frequency spectrum in a predetermined frequency band to approximate the frequency spectrum with a linear expression (regression line), thereby calculating the pre-correction feature value that characterizes the approximated linear expression. For example, in the case of the frequency spectrum C₁ illustrated in FIG. 5, the approximating unit 333a executes regression analysis in the frequency band F and approximates the frequency spectrum C₁ with a linear expression, thereby obtaining a regression line L₁₀. In other words, the approximating unit 333a calculates, as the pre-correction feature values, a slope a₀ of the regression line L₁₀, an intercept b₀, and mid-band fit c₀=a₀f_{M}+b₀ that is a value on the regression line at the center frequency f_{M}=(f_{L}+f_{H})/2 of the frequency band F.

Among the three sets of the pre-correction feature values, it is considered that the slope a₀ is correlated to the size of a scattering substance for ultrasound waves and, generally, the larger the scattering substance is, the smaller the value of the slope. Furthermore, the intercept b₀ is correlated to the size of a scattering substance, a difference in the acoustic impedance, the number density (concentration) of a scattering substance, or the like. Specifically, it is considered that the larger the scattering substance is, the larger the value of the intercept b₀ is, the larger the difference in the acoustic impedance, the larger the value is, and the larger the number density of the scattering substance is, the larger the value is. The mid-band fit c₀ is an indirect parameter derived from the slope a₀ and the intercept b₀, and it applies an intensity of the spectrum at the center of a valid frequency band. Therefore, it is considered that the mid-band fit c₀ is somewhat correlated to the luminance of a B-mode image in addition to the size of the scattering substance, a difference in the acoustic impedance, and the number density of the scattering substance. Moreover, the feature-value calculating unit 333 may approximate a frequency spectrum by using regression analysis with a polynomial of the second or more degrees.

Correction performed by the attenuation correcting unit 333b is explained. Generally, attenuation A(f,z) of ultrasound waves is attenuation that occurs while ultrasound waves move back and forth between a receive depth 0 and the receive depth z, and it is defined as a change (a difference in decibel representation) in the intensity before and after a back-and-forth movement. It is experimentally known that the attenuation A(f,z) is proportional to a frequency in uniform tissue, and it is represented by the following Equation (1).

A(f,z)=2αzf (1)

Here, the proportional constant α is a value called an attenuation rate. Furthermore, z is the receive depth of an ultrasound wave, and f is a frequency. When the observation target is a living body, the specific value of the attenuation rate α is determined depending on a site of the living body. The unit of the attenuation rate α is, for example, dB/cm/MHz. Moreover, according to the present embodiment, a configuration may be such that the value of the attenuation rate α is changeable by an input from the input unit 35.

The attenuation correcting unit 333b conducts attenuation correction on pre-correction feature values (the slope a₀, the intercept b₀, the mid-band fit c₀) extracted by the approximating unit 333a in accordance with Equations (2) to (4) described below, thereby calculating feature values a, b, c.

a=a₀+2αz (2)

b=b₀ (3)

c=c₀+A(f_{M},z)=c₀+2αzf_{M}(=af_{M}+b) (4)

As it is understood by Equations (2), (4), the attenuation correcting unit 333b conducts correction such that the amount of correction is larger as the receive depth z of an ultrasound wave is larger. Furthermore, according to Equation (3), correction with regard to the intercept is identify transformation. This is because the intercept is a frequency component corresponding to the frequency 0 (Hz) and it is not affected by attenuation.

FIG. 6 is a graph that illustrates a straight line having, as parameters, the feature values a, b, c calculated by the attenuation correcting unit 333b. The equation for a straight line L₁ is represented by:

I=af+b=(a₀+2αz)f+b₀ (5).

As it is understood by Equation (5), the straight line L₁ has a larger slope (a>a₀) and the same intercept (b=b₀) as compared with the straight line L₁₀ before attenuation correction.

The representative-value calculating unit 334 generates a histogram representing the frequency of the target feature value to be displayed among the feature values a, b, c, calculated by the feature-value calculating unit 333 at each sample point, and calculates a representative value of the feature values in each region of interest from the generated histogram. According to the first embodiment, the average value of the feature values c in each region of interest is calculated from each histogram, and it is set as a representative value.

The threshold setting unit 335 sets a threshold based on the representative value in each region of interest, calculated by the representative-value calculating unit 334. The threshold is a value representing the feature value, and it is a value for determining the boundary between color phases in a color pattern on a feature-value image. According to the first embodiment, the threshold setting unit 335 selects a smaller representative value between two representative values and sets the maximal value in the region of interest as a threshold.

The display-specification setting unit 336 sets the display specification of the target feature value to be displayed on the display device 4 based on the threshold set by the threshold setting unit 335. Specifically, according to the first embodiment, the display-specification setting unit 336 sets the color pattern of color phases, which is the display specification of the feature value c, based on the threshold.

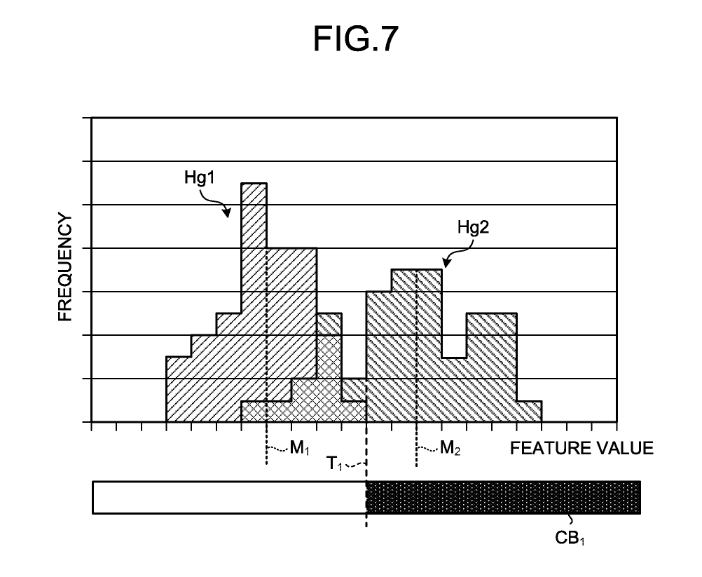

FIG. 7 is a diagram that illustrates a process performed by the display-specification setting unit 336 in the ultrasound observation device 3 according to the first embodiment. In FIG. 7, the horizontal axis is the feature value c. Furthermore, in FIG. 7, the vertical axis is the frequency of the feature value c. FIG. 7 is a graph representing the distribution of the feature value c and the frequency.

Here, as illustrated in FIG. 7, the distributions of the feature value c in regions of interest are different depending on the type of a characteristic of living tissue (hereafter, referred to as tissue characteristic). For example, when the feature value c exhibits a certain tissue characteristic, it is distributed in a histogram Hg1 and, when it exhibits a different tissue characteristic, it is distributed in a histogram Hg2. Here, when the feature value c is represented in the same color, there is a possibility that a difference between two tissue characteristics, e.g., a normal region and an abnormal region of the same tissue, is not clearly represented.

According to the first embodiment, the display-specification setting unit 336 sets the display specification of the target feature value c to be displayed based on the set threshold. Specifically, the representative-value calculating unit 334 first generates the histograms Hg1, Hg2 of the feature values in the respective regions of interest, obtains average values M₁, M₂ in the respective regions of interest, and sets the average values M₁, M₂ as representative values in the respective regions of interest. Then, the threshold setting unit 335 selects the smaller representative value (the average value M₁ in FIG. 7) between the average values M₁, M₂, and sets the maximal value in the histogram (the histogram Hg1 in FIG. 7) having the average value as a threshold (a threshold T₁ in FIG. 7). The display-specification setting unit 336 sets, as a display specification, a color bar CB₁ in which a color phase is changed at the set threshold (the threshold T₁) as a boundary. In FIG. 7, the display specification is such that, with the threshold T₁ as a boundary, the side of smaller feature value is colored in red and the side of larger feature value is colored in blue. Here, in the color bar CB₁ illustrated in FIG. 7, the red-colored region is illustrated in white, and the blue-colored region is illustrated by hatching. This allows tissue characteristics (including the normal and the abnormal in the same tissue), which are included in two regions of interest and are different from each other, to be displayed clearly and distinctively.

The image processing unit 34 includes: a B-mode image data generating unit 341 that generates B-mode image data, which is an ultrasound image displayed after the amplitude of an echo signal is converted into luminance; and a feature-value image data generating unit 342 that generates feature-value image data that is displayed together with a B-mode image by relating the feature value calculated by the attenuation correcting unit 333b to visual information.

The B-mode image data generating unit 341 performs signal processing on B-mode receive data received from the signal processing unit 32 by using a known technique, such as gain processing, contrast processing, or γ correction processing and decimates data, or the like, in accordance with the data step width defined depending on the display range of an image on the display device 4, thereby generating B-mode image data. B-mode images are gray-scaled images in which the values of R (red), G (green), B (blue), which are variables when the RGB color system is adopted as a color space, are matched.

The B-mode image data generating unit 341 performs coordinates conversion on B-mode receive data from the signal processing unit 32 to rearrange a scan area so as to be properly represented in space and then performs an interpolation process on B-mode receive data sets to fill gaps in the B-mode receive data sets, thereby generating B-mode image data. The B-mode image data generating unit 341 outputs the generated B-mode image data to the feature-value image data generating unit 342.

The feature-value image data generating unit 342 superimposes visual information related to the feature value calculated by the feature-value calculating unit 333 on each pixel in the image of the B-mode image data, thereby generating feature-value image data. The feature-value image data generating unit 342 assigns the visual information that corresponds to the feature value on the frequency spectrum calculated from the sample data group Fⱼ (j=1, 2, . . . , K), illustrated in for example FIG. 4, to the pixel area that corresponds to the amount of data on the single sample data group Fⱼ. The feature-value image data generating unit 342 relates the color phase, which is visual information, to any one of, for example, the slope, the intercept, and the mid-band fit, described above, thereby generating a feature-value image. Specifically, when the feature-value image data generating unit 342 relates a color phase, which is visual information, to the feature value c, the visual information is assigned based on the color pattern set by the display-specification setting unit 336. The visual information related to the feature value may include, as well as the color phase, for example, color saturation, brightness value, luminance value, or a variable in a color space forming a predetermined color system, e.g., R (red), G (green), or B (blue).

Furthermore, when the feature-value image data generating unit 342 performs gain adjustment or contrast processing, the visual information (luminance value) may be adjusted independently from the gain adjustment performed by the B-mode image data generating unit 341, or a luminance difference may be adjusted independently from the contrast of B-mode image data. An adjustment value may be set depending on each type of the ultrasound endoscope 2.

Furthermore, when the feature-value image data generating unit 342 conducts γ correction, the same correction table as that for γ correction performed by the B-mode image data generating unit 341 may be used, or a different correction table may be used. The curvature of the γ curve for γ correction and the ratio of an input to an output may be adjusted depending on each type of the ultrasound endoscope 2.

The control unit 36 is implemented by using a CPU, various types of arithmetic circuits, or the like, having calculation and control functions. The control unit 36 reads information, saved and stored in the storage unit 37, from the storage unit 37 and performs various types of arithmetic processing related to the method for operating the ultrasound observation device 3, thereby controlling the ultrasound observation device 3 in an integrated manner. Furthermore, the control unit 36 may be configured by using the CPU, or the like, shared by the signal processing unit 32 and the calculating unit 33.

The control unit 36 includes a region-of-interest setting unit 361 that sets the region of interest in accordance with a command input received by the input unit 35. The region-of-interest setting unit 361 sets a region of interest based on the setting input (command point) input via for example the input unit 35. The region-of-interest setting unit 361 may arrange a frame having a predetermined shape based on the position of the command point or form a frame by connecting the point group of multiple input points. Furthermore, when a region of interest is set by using a keyboard, the region-of-interest setting unit 361 may be capable of switching a region of interest for measurement, which is circular (including an ellipse), and a region of interest for observation, which is rectangular or a fan-like shape, due to a key operation received by the input unit 35, e.g., operation (press) on the R key or the T key. In addition, the region-of-interest setting unit 361 may assign deletion of a region of interest to any of the keys so as to delete the selected region of interest due to an operation on the key. When a region of interest for measurement is set, the region-of-interest setting unit 361 may perform control so as to display the target region to be measured in white. Moreover, the region-of-interest setting unit 361 may perform control so as to prevent a region of interest from being set in the image area that corresponds to the sound ray at the outermost edge side of the ultrasound transducer 21, e.g., at both edge sides in a scanning direction, when the ultrasound transducer is of a convex type.

The storage unit 37 stores multiple sets of the feature values calculated by the attenuation correcting unit 333b for each frequency spectrum and image data generated by the image processing unit 34. Furthermore, the storage unit 37 includes a display-specification information storage unit 371 that stores the setting for calculating a representative value, the condition for setting a threshold, and the condition for setting a color pattern.

In addition to the ones described above, the storage unit 37 stores, for example, information needed for an amplification process (the relationship between an amplification factor and a receive depth illustrated in FIG. 2), information needed for an amplification correction process (the relationship between an amplification factor and a receive depth illustrated in FIG. 3), information needed for an attenuation correction process (see Equation (1)), or information on a window function (Hamming, Hanning, Blackman, or the like) needed for a frequency analysis process.

Furthermore, the storage unit 37 stores various programs including an operation program to implement a method for operating the ultrasound observation device 3. The operation program may be widely distributed by being recorded in a recording medium readable by a computer, such as hard disk, flash memory, CD-ROM, DVD-ROM, or flexible disk. Furthermore, the above-described various programs may be acquired by being downloaded via a communication network. The communication network mentioned here is implemented by using, for example, an existing public network, LAN (Local Area Network), or WAN (Wide Area Network), and it may be wired or wireless.

The storage unit 37 having the above configuration is implemented by using a ROM (Read Only Memory) having various programs, or the like, previously installed therein, a RAM (Random Access Memory) storing calculation parameters, data, and the like, for each process, or the like.

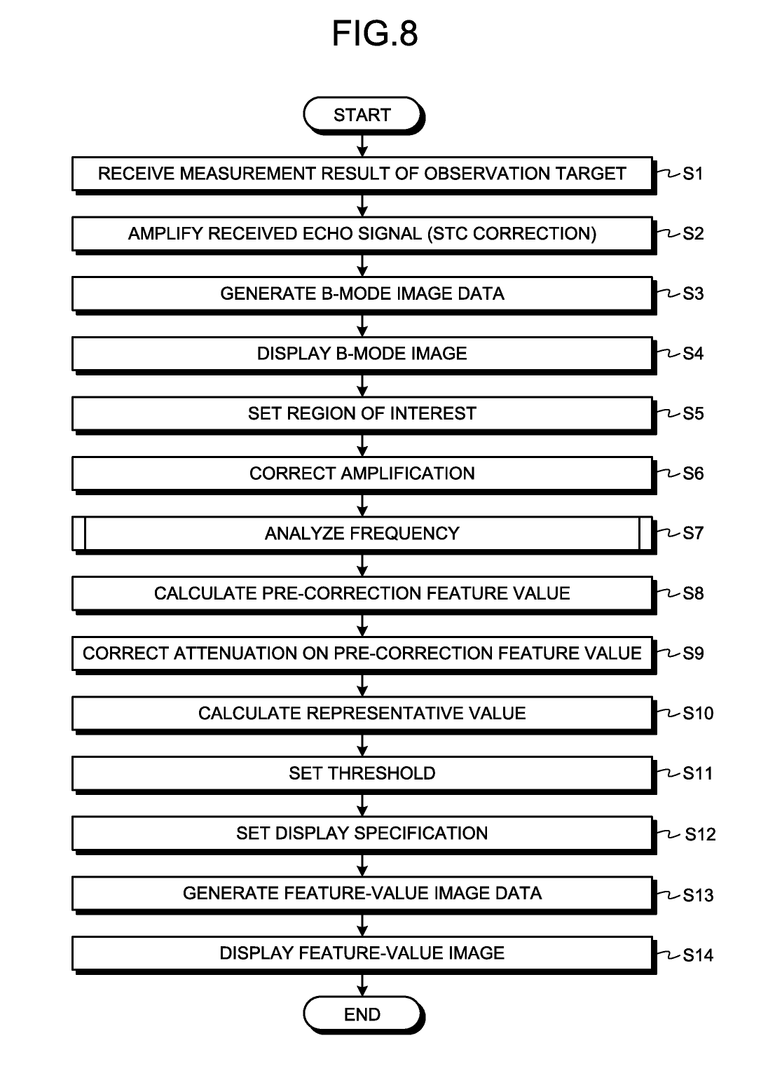

FIG. 8 is a flowchart that illustrates the outline of a process performed by the ultrasound observation device 3 having the above configuration. First, the ultrasound observation device 3 receives an echo signal, which is a measurement result of the observation target by the ultrasound transducer 21 from the ultrasound endoscope 2 (Step S1).

After receiving an echo signal from the ultrasound transducer 21, the signal amplifying unit 311 amplifies the echo signal (Step S2). Here, the signal amplifying unit 311 amplifies (STC correction) the echo signal based on the relationship between the amplification factor and the receive depth illustrated in for example FIG. 2.

Then, the B-mode image data generating unit 341 generates B-mode image data by using the echo signal amplified by the signal amplifying unit 311 and outputs it to the display device 4 (Step S3). After receiving the B-mode image data, the display device 4 displays the B-mode image that corresponds to the B-mode image data (Step S4).

Then, the region-of-interest setting unit 361 sets a region of interest based on the setting input via the input unit 35 (Step S5: a region-of-interest setting step).

The amplification correcting unit 331 conducts amplification correction on a signal output from the transmitting/receiving unit 31 such that the amplification factor is constant regardless of the receive depth (Step S6). Here, the amplification correcting unit 331 conducts amplification correction such that the relationship between the amplification factor and the receive depth illustrated in for example FIG. 3 is satisfied.

Then, the frequency analyzing unit 332 conducts frequency analysis using FFT calculation, thereby calculating a frequency spectrum for the entire sample data group (Step S7: frequency analysis step). FIG. 9 is a flowchart that illustrates the outline of the process performed by the frequency analyzing unit 332 at Step S7. With reference to the flowchart illustrated in FIG. 9, a frequency analysis process is explained below in detail.

First, the frequency analyzing unit 332 sets a counter k for identifying the target sound ray to be analyzed to k₀ (Step S21).

Then, the frequency analyzing unit 332 sets a default value Z^{(k)}₀ at the position of data (corresponds to the receive depth) Z^{(k)}, which is representative of the sequential data group (sample data group) acquired for FFT calculation (Step S22). For example, FIG. 4 illustrates a case where the position of the eighth data of the sound ray SRₖ is set as the default value Z^{(k)}₀, as described above.

Then, the frequency analyzing unit 332 acquires the sample data group (Step S23) and applies the window function stored in the storage unit 37 to the acquired sample data group (Step S24). By thus applying the window function to the sample data group, it is possible to prevent the sample data group from being discontinuous at a boundary and to prevent the occurrence of artifacts.

Then, the frequency analyzing unit 332 determines whether the sample data group at the data position Z^{(k)} is a normal data group (Step S25). As described with reference to FIG. 4, a sample data group needs to have the number of sets of data that is a power of two. Hereafter, the number of sets of data in a normal sample data group is 2ⁿ (n is a positive integer). According to the present embodiment, the data position Z^{(k)} is set as close to the center of the sample data group, to which Z^{(k)} belongs, as possible. Specifically, as the number of sets of data in a sample data group is 2ⁿ, Z^{(k)} is set at the 2ⁿ/2 (=2ⁿ⁻¹)^{th} position that is close to the center of the sample data group. In this case, a normal sample data group means that there are 2ⁿ⁻¹−1 (=N) sets of data before the data position Z^{(k)} and there are 2ⁿ⁻¹ (=M) sets of data after the data position Z^{(k)}. In the case illustrated in FIG. 4, the sample data groups F₁, F₂, F₃, . . . , F_{K-1} are all normal. Here, FIG. 4 illustrates a case where n=4 (N=7, M=8).

As a result of the determination at Step S25, when the sample data group at the data position Z^{(k)} is normal (Step S25: Yes), the frequency analyzing unit 332 proceeds to Step S27 described later.

As a result of the determination at Step S25, when the sample data group at the data position Z^{(k)} is faulty (Step S25: No), the frequency analyzing unit 332 inserts zero data corresponding to a shortage, thereby generating a normal sample data group (Step S26). A window function is applied to a sample data group (e.g., the sample data group F_{K} in FIG. 4), which is determined to be faulty at Step S25, before zero data is added. Therefore, insertion of zero data to the sample data group does not cause the data to be discontinuous. After Step S26, the frequency analyzing unit 332 proceeds to Step S27 described later.

At Step S27, the frequency analyzing unit 332 conducts FFT calculation by using a sample data group, thereby obtaining a frequency spectrum that is the frequency distribution of the amplitude (Step S27).

Then, the frequency analyzing unit 332 changes the data position Z^{(k)} by a step width D (Step S28). The step width D is previously stored in the storage unit 37. FIG. 4 illustrates a case where D=15. It is preferable that the step width D matches the data step width used by the B-mode image data generating unit 341 to generate B-mode image data; however, to reduce the amount of calculations by the frequency analyzing unit 332, a value larger than the data step width may be set as the step width D.

Then, the frequency analyzing unit 332 determines whether the data position Z^{(k)} is larger than a maximal value Z^{(k)}ₘₐₓ in the sound ray SRₖ (Step S29). When the data position Z^{(k)} is larger than the maximal value Z^{(k)}ₘₐₓ (Step S29: Yes), the frequency analyzing unit 332 increments the counter k by 1 (Step S30). This means that the process proceeds to the next sound ray. Conversely, when the data position Z^{(k)} is equal or less than the maximal value Z^{(k)}ₘₐₓ(Step S29: No), the frequency analyzing unit 332 returns to Step S23. In this manner, the frequency analyzing unit 332 conducts FFT calculation on the sample data group of [(Z^{(k)}ₘₐₓ−Z^{(k)}₀+1)/D+1] sets with regard to the sound ray SRₖ. Here, [X] represents the largest integer less than X.

After Step S30, the frequency analyzing unit 332 determines whether the counter k is larger than the maximal value kₘₐₓ (Step S31). When the counter k is larger than the maximal value kₘₐₓ (Step S31: Yes), the frequency analyzing unit 332 terminates the sequential frequency analysis process. Conversely, when the counter k is less than the maximal value kₘₐₓ (Step S31: No), the frequency analyzing unit 332 returns to Step S22. The maximal value kₘₐₓ is a value of an optional command input via the input unit 35 by a user such as an operator or a value previously set in the storage unit 37.

In this manner, the frequency analyzing unit 332 performs FFT calculations multiple times on each of (kₘₐₓ−k₀+1) sound rays within the target area to be analyzed. A result of the FFT calculation is stored in the storage unit 37 together with the receive depth and the receive direction.

Furthermore, in the above explanation, the frequency analyzing unit 332 performs a frequency analysis process on all the areas for which ultrasound signals have been received; however, a frequency analysis process may be performed only in the set region of interest.

Subsequent to the above-described frequency analysis process at Step S7, the feature-value calculating unit 333 calculates a pre-correction feature value on each of frequency spectra and conducts attenuation correction, which is to remove effects of attenuation of ultrasound waves, on the pre-correction feature value on each frequency spectrum, thereby calculating a corrected feature value on each frequency spectrum (Steps S8 to S9: feature-value calculation step).

At Step S7, the approximating unit 333a conducts regression analysis on each of frequency spectra generated by the frequency analyzing unit 332, thereby calculating a pre-correction feature value that corresponds to each frequency spectrum (Step S8). Specifically, the approximating unit 333a conducts regression analysis on each frequency spectrum to execute approximation with a linear expression and, as a pre-correction feature value, calculates the slope a₀, the intercept b₀, and the mid-band fit c₀. For example, the straight line L₁₀ illustrated in FIG. 5 is a regression line that is obtained when the approximating unit 333a conducts approximation on the frequency spectrum C₁ in the frequency band F due to regression analysis.

Then, the attenuation correcting unit 333b conducts attenuation correction by using the attenuation rate α on the pre-correction feature value that is obtained when the approximating unit 333a executes approximation on each frequency spectrum so as to calculate the corrected feature value and stores the calculated corrected feature value in the storage unit 37 (Step S9). The straight line L₁ illustrated in FIG. 6 is an example of the straight line obtained when the attenuation correcting unit 333b performs an attenuation correction process.

At Step S9, the attenuation correcting unit 333b executes calculation by substituting the data position Z=(fₛₚ/2vₛ)Dn obtained by using the data array of the sound ray of an ultrasound signal into the receive depth z in the above-described Equations (2), (4). Here, fₛₚ is the sampling frequency of data, vₛ is the sound velocity, D is the step width, and n is the number of data steps from the first set of data in the sound ray to the data position in the target sample data group to be processed. For example, when the sampling frequency fₛₚ of data is 50 MHz, the sound velocity vₛ is 1530 m/sec, and the step width D is 15 when the data array illustrated in FIG. 4 is adopted, z=0.2295n (mm).

Then, the display specification of the target feature value to be displayed, included in the feature value calculated at Step S8, is set with regard to each pixel of the B-mode image data generated by the B-mode image data generating unit 341 (Step S10 to Step S12).

First, at Step S10, the representative-value calculating unit 334 generates the histogram of a feature value in each region of interest, obtains the average value of each region of interest, and sets the average value as the representative value of the region of interest. For example, in the case of FIG. 7, the representative-value calculating unit 334 generates the histograms Hg1, Hg2 of the feature values in the respective regions of interest, obtains the average values M₁, M₂ of the respective regions of interest, and sets the average values M₁, M₂ as representative values of the respective regions of interest (representative-value calculation step).

At Step S11 that follows Step S10, the threshold setting unit 335 selects a smaller representative value among the representative values of the respective regions of interest and sets the maximal value in the histogram having the average value as a threshold. For example, in the case of FIG. 7, the threshold setting unit 335 selects a smaller representative value (the average value M₁) among the average values M₁, M₂ and sets the maximal value in the histogram Hg1 having the average value M₁ as the threshold T₁ (threshold setting step).

At Step S12 that follows Step S11, the display-specification setting unit 336 sets, as a display specification, the color pattern in which color phases are changed at the set threshold as a boundary. For example, in the case of FIG. 7, the display-specification setting unit 336 sets the color condition in which, with the threshold T₁ as a boundary, the side of smaller values is in red and the side of larger values is in blue (display-specification setting step).

The feature-value image data generating unit 342 generates feature-value image data by superimposing visual information related to the feature value calculated at Step S8 on each pixel of the B-mode image data generated by the B-mode image data generating unit 341 in accordance with the coloring condition set at Step S12 (Step S13: feature-value image data generation step).

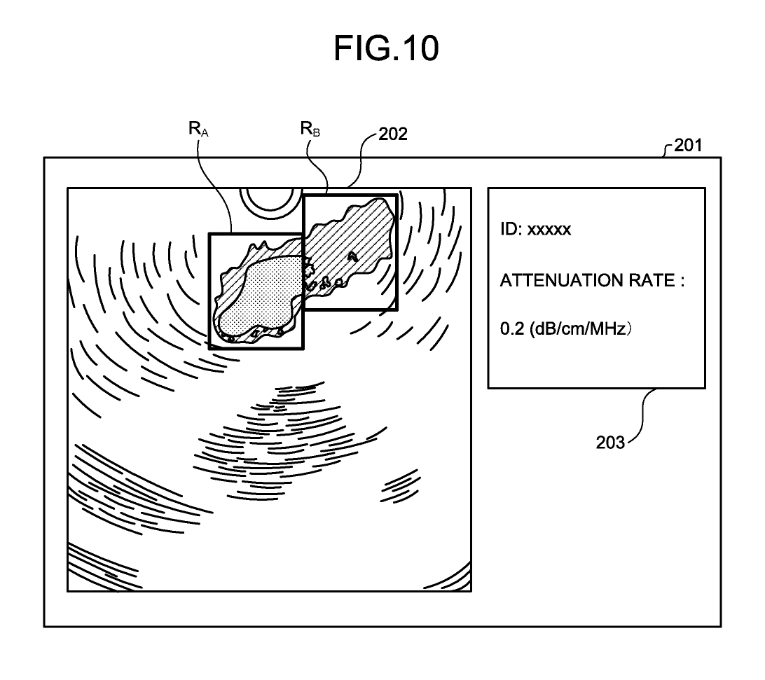

Then, the display device 4 displays the feature-value image that corresponds to the feature-value image data generated by the feature-value image data generating unit 342 under the control of the control unit 36 (Step S14). FIG. 10 is a diagram that schematically illustrates a display example of the feature-value image on the display device 4. A feature-value image 201 illustrated in the drawing includes: a superimposed-image display section 202 that displays an image where visual information related to the feature value is superimposed on the B-mode image; and an information display section 203 that displays identification information, and the like, on the observation target. In FIG. 10, two regions of interest (regions of interest R_{A}, R_{B}) are set on the feature-value image 201, and a color is used in accordance with the feature value.

The information display section 203 may further display information on the feature value, information on an approximate expression, image information such as gain or contrast, and the like. Furthermore, the B-mode image that corresponds to feature-value image may be displayed alongside the feature-value image.

Furthermore, on a feature-value image, for example, a sound point that is determined to be noise due to difficulty in calculation of the feature value may be displayed in gray or black. Moreover, the sound point that is determined to be noise is excluded from the calculation target when the average or the standard deviation of the feature value is calculated.

Furthermore, when a command for storing in the storage unit 37 is input while an ultrasound image is displayed, unprocessed raw data, on which signal processing has not been performed, may be stored in the storage unit 37. Moreover, in explanation of the flowchart illustrated in FIG. 8, a frequency feature value is calculated based on an acquired echo signal and a feature-value image is generated; however, the frequency feature value or the feature value image may be generated by using raw data stored in the storage unit 37.

Furthermore, RF data stored in the storage unit 37 and selected by a user may be read and a B-mode image and a feature-value image generated based on the RF data may be generated and displayed. Here, the B-mode image data generating unit 341 first generates B-mode image data based on the read RF data or the RF data for B-mode image generation, corresponding to the RF data, and the display device 4 displays the B-mode image. Then, when the setting of a region of interest is input, the feature-value image data generating unit 342 generates the visual information related to the feature value with regard to the region of interest and generates feature-value image data where the visual information is superimposed on the B-mode image data. The display device 4 displays the feature-value image that corresponds to the generated feature-value image data.

According to the first embodiment described above, colors are changed with regard to the distributions of the feature value in two different regions of interest by using a threshold that is set based on the histograms; thus, tissue characteristics in multiple regions of interest may be represented clearly and distinctively.

Furthermore, although a representative value and a histogram are generated by using the feature value c in explanation of the above-described first embodiment, the used feature value is different depending on the target feature value to be displayed. For example, the feature value a or the feature value b described above are sometimes used, or the sound velocity or the degree of hardness calculated as a feature value is sometimes used. Furthermore, the representative-value calculating unit 334 may generate a histogram based on the frequency of the summed value of multiple sets of feature values, e.g., the frequency of the sum of the feature value a and the feature value c. Moreover, the feature values may be not only the above-described feature values a, b, c, which are frequency feature values, but also the degree of hardness, the sound velocity, or the like.

Furthermore, although the representative-value calculating unit 334 sets the average value of the selected histogram as a representative value in explanation of the above-described first embodiment, this is not a limitation. For example, the middle value or the mode value may be set as a representative value.

Furthermore, although the threshold setting unit 335 sets the maximal value of the selected histogram as a threshold in explanation of the above-described first embodiment, this is not a limitation. For example, any of the average value, the middle value, the mode value, the standard deviation, and the minimum value, or the value of the combination of any two or more of them, e.g., the value of sum of the average value and the standard deviation, may be set as a threshold.

Furthermore, although the threshold setting unit 335 sets a threshold by using the histogram that corresponds to a smaller representative value in explanation of the above-described first embodiment, a threshold may be set by using the histogram that corresponds to a larger representative value. In this case, for example, the threshold setting unit 335 sets the minimum value in the selected histogram as a threshold or sets the value of subtraction of the standard deviation from the average value as a threshold.

Furthermore, although a representative value is calculated in association with calculation of the feature value, a threshold is set, and a display specification is set in explanation of the above-described first embodiment, this is not a limitation. For example, the display-specification information storage unit 371 may store the previously set color condition and, without calculating the above-described representative value, or the like, the display-specification setting unit 336 may read the selected color condition from the display-specification information storage unit 371 and set it in accordance with an input from the user.

Furthermore, although representative values are calculated with regard to the two set regions of interest, a threshold is set, and a display specification is set in explanation of the above-described first embodiment, the regions of interest are not always two, and three or more may be set. For example, when three regions of interest are set, the representative-value calculating unit 334 calculates a representative value in each region of interest, the threshold setting unit 335 sets a threshold based on the representative value, and the display-specification setting unit 336 sets a display specification. Here, for example, the threshold setting unit 335 sets, as a threshold, the maximal value in each of the two regions of interest that correspond to the two representative values other than the maximum representative value among the three representative values. The display-specification setting unit 336 assigns different color phases to the ranges of the feature value divided by the two set thresholds as boundaries, thereby setting the display specification.

Furthermore, the representative-value calculating unit 334 generates a histogram and also calculates a representative value in explanation of the above-described first embodiment; however, when deviation, and the like, is not used, e.g., when the representative value is an average value and the threshold is a maximal value, a histogram does not need to be generated.

Furthermore, a B-mode image and a feature-value image that are displayed live are generated as in the flowchart illustrated in FIG. 8 in explanation of the above-described first embodiment; however, a B-mode image and a feature-value image are sometimes displayed to be freeze in accordance with input of a freeze command. Here, it is possible to make the setting such that the target feature value to be calculated is changed, e.g., the feature value a is changed to the feature value c, in accordance with selection between the live display and the freeze display.

Moreover, in the above-described first embodiment, the generated histogram may be displayed together with a feature-value image after the setting position and the range of the region of interest are confirmed, or a feature-value image (visual information) may be displayed as a result of calculation before the settings of the region of interest are confirmed.

### Modification 1 of the First Embodiment

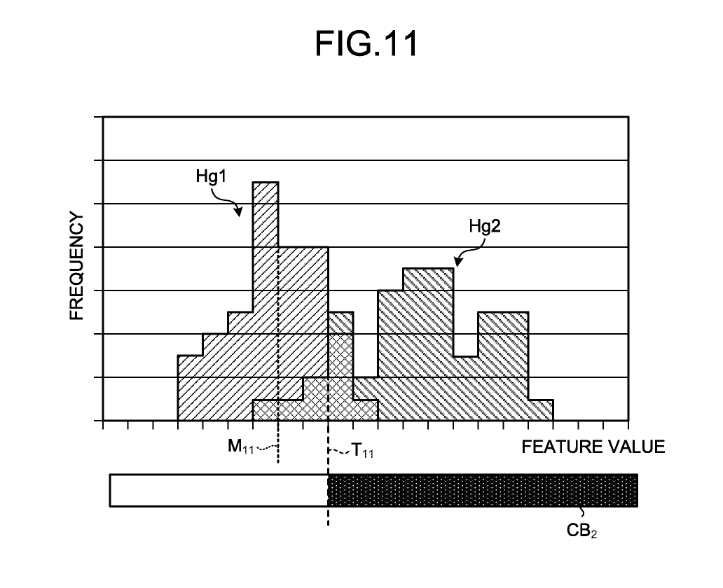

FIG. 11 is a diagram that illustrates a process performed by the display-specification setting unit in the ultrasound observation device according to the modification 1 of the first embodiment. According to the modification 1, the threshold setting unit 335 sets, as a threshold, the value that is obtained by adding the doubled value of the standard deviation to the average value. Specifically, the threshold setting unit 335 first obtains the standard deviation of the histogram with regard to the histogram Hg1 for which it is determined that a value is smaller. Then, the threshold setting unit 335 calculates a threshold T₁₁ according to T₁₁=M₁₁+2σ where the average value of the histogram Hg1 is M₁₁, the standard deviation is σ, and the threshold is T₁₁. The display-specification setting unit 336 sets a color bar CB₂ in which color phases are changed at the set threshold as a boundary. Here, in the color bar CB₂ illustrated in FIG. 11, the red-colored area is illustrated in white, and the blue-colored area is illustrated by hatching.

### Modification 2 of the First Embodiment

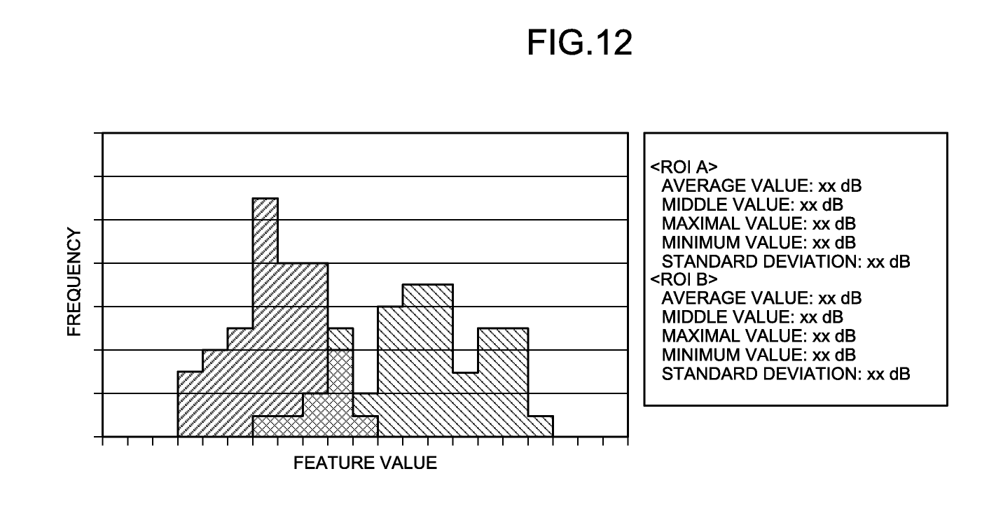

FIG. 12 is a diagram that schematically illustrates an example of the display on the display device in the ultrasound observation device according to the modification 2 of the first embodiment. As illustrated in FIG. 12, a histogram or information (the average value, the middle value, the maximal value, the minimum value, and the standard deviation in FIG. 12) related to each region of interest (ROI A, ROI B) may be displayed on the feature-value image illustrated in FIG. 10. The above-described information is a value obtained from the histogram generated by using a value in each region of interest. By displaying the information illustrated in FIG. 12, the user is capable of setting a representative value or a threshold by referring to the values. Furthermore, the above-described display example is only an example, and it is possible to make the setting such that any one or more of the average value, the middle value, the maximal value, the minimum value, and the standard deviation are displayed.

Furthermore, according to the above-described modification 2, the scale (scale marks) of a histogram may be automatically set, may be set as a predetermined fixed value (interval), or it is possible to make the setting, either automatic setting or fixed-value setting.

### Second Embodiment

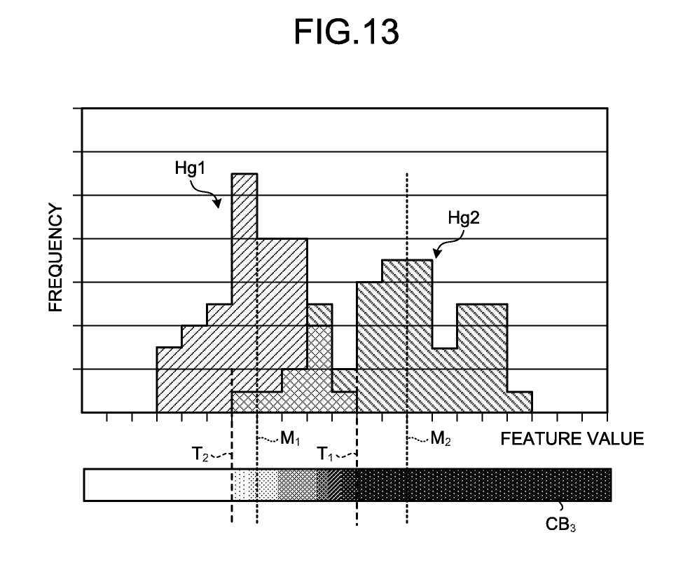

FIG. 13 is a diagram that illustrates a process performed by a display-specification setting unit in the ultrasound observation device according to the second embodiment. In explanation of the above-described first embodiment, the threshold setting unit 335 sets a single threshold; however, according to the second embodiment, two thresholds are set based on the feature value of each histogram. In this explanation, the configuration of the ultrasound observation system is the same as that of the above-described ultrasound observation system 1.

The threshold setting unit 335 sets two thresholds based on the representative value of each region of interest calculated by the representative-value calculating unit 334. According to the second embodiment, the threshold setting unit 335 determines the magnitude relationship between the two representative values and sets two thresholds in accordance with a determination result. With regard to the histogram (the histogram Hg1 in FIG. 13) having the representative value that is determined to be a smaller value among the average values M₁, M₂, the threshold setting unit 335 sets for example the maximal value of the histogram Hg1 as a first threshold (the threshold T₁ in FIG. 13). Furthermore, with regard to the histogram (the histogram Hg2 in FIG. 13) having the representative value that is determined to be a larger value among the average values M₁, M₂, the threshold setting unit 335 sets for example the minimum value of the histogram Hg2 as a second threshold (the threshold T₂ in FIG. 13).

The display-specification setting unit 336 sets the display specification of the target feature value to be displayed on the display device 4 based on the first and the second thresholds set by the threshold setting unit 335. Specifically, as the display specification of the feature value c, the display-specification setting unit 336 sets a color bar CB₃ in which blue is set for more than the first threshold, red is set for less than the second threshold, and color phases are gradually changed between the first threshold and the second threshold. In the interval between the first threshold and the second threshold, colors (color phases) having different light wavelengths are arranged in a continuous manner (including a multistep manner). Specifically, from the left, there are red, orange, yellow, green, and blue (indigo) in descending order of a wavelength of visible light. For example, the longest wavelength is 750 nm, which is the same as that of color that is more than the first threshold, and the shortest wavelength is 500 (445) nm, which is the same as that of color that is more than the second threshold. Here, in the color bar CB₃ illustrated in FIG. 13, the red-colored area is illustrated in white, and a shorter light wavelength corresponding to a color is illustrated by darker hatching (having a larger painted area). In explanation of the second embodiment, the display specification is set such that color phases are continuously changed between the first threshold and the second threshold; however, the interval between the first threshold and the second threshold may be displayed in a color phase different from a first color phase for more than the first threshold and a second color phase for less than the second threshold, and it may be colored with a single color phase that is different from the color phases. The “different color phase” mentioned here refers to the color phase that corresponds to a wavelength different from the wavelengths that correspond to the first and the second color phases.

According to the second embodiment, the display-specification setting unit 336 sets the display specification of the target feature value to be displayed based on the set first and second thresholds. The display-specification setting unit 336 sets, as the display specification, a color pattern in which color phases are changed at the set first and second thresholds (the thresholds T₁, T₂) as boundaries. In the display specification of FIG. 13, the side of larger feature value with the threshold T₁ as a boundary is colored in blue, the side of smaller feature value with the threshold T₂ as a boundary is colored in red, and the color phases are gradually changed in the interval between the thresholds T₁, T₂ (the region where the histograms Hg1, Hg2 are overlapped). This makes it possible to clearly distinguish between different tissue characteristics (including the normal and the abnormal in the same tissue) that are included in two respective regions of interest and makes it possible to identify, for example, the transition area between tissue characteristics.

### Third Embodiment

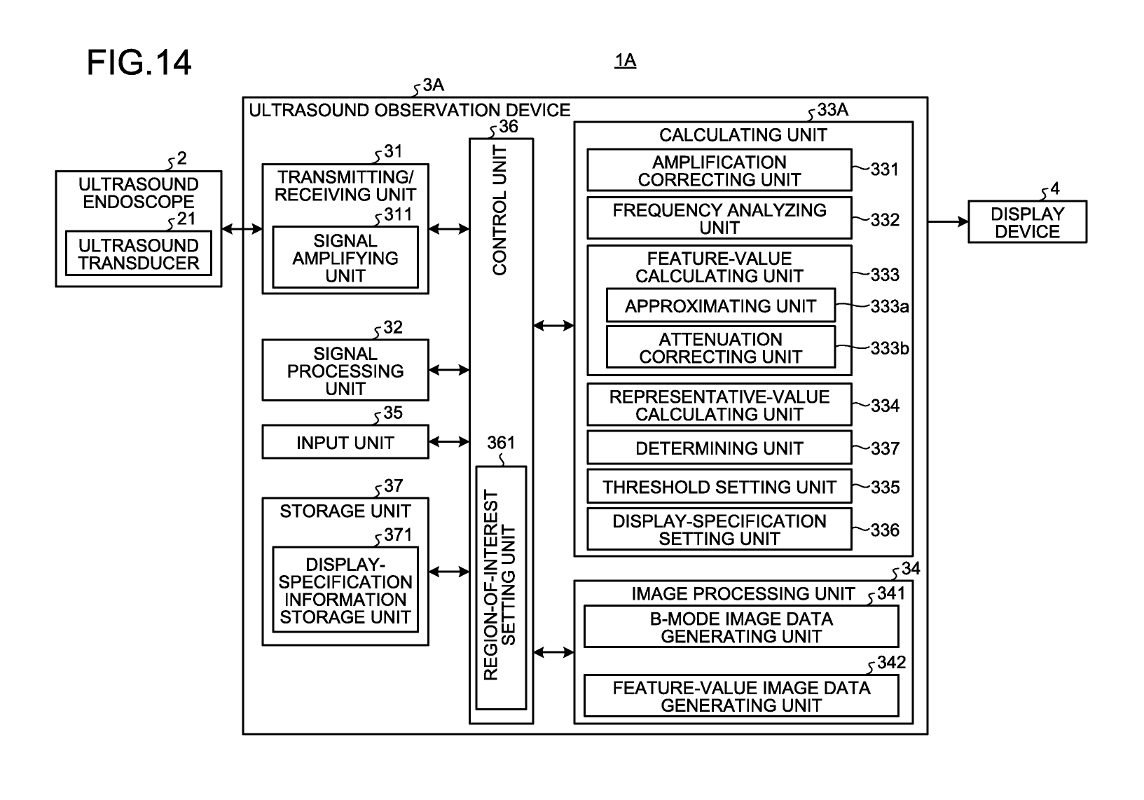

FIG. 14 is a block diagram that illustrates a configuration of an ultrasound observation system 1A including an ultrasound observation device 3A according to a third embodiment. The ultrasound observation system 1A illustrated in the drawing includes the ultrasound observation device 3A instead of the ultrasound observation device 3 in the ultrasound observation system 1 according to the above-described first embodiment. According to the third embodiment, it is determined whether there is an overlapped area in the histograms of the respective regions of interest, and a threshold is set based on a determination result. A calculating unit 33A in the ultrasound observation device 3A includes a determining unit 337 in addition to the configuration of the above-described calculating unit 33. The other configuration is the same as that of the ultrasound observation system 1 according to the first and the second embodiments.

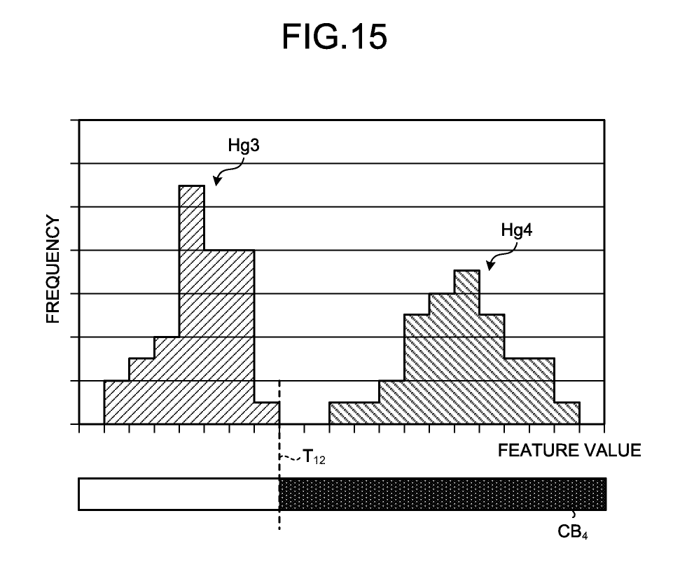

FIG. 15 is a diagram that illustrates a process performed by the display-specification setting unit 336 in the ultrasound observation device 3A according to the third embodiment. The representative-value calculating unit 334 generates histograms Hg3, Hg4 with regard to the two set regions of interest in the same manner as in the above-described first and second embodiments.

The determining unit 337 determines whether there is an overlapped area in the histograms Hg1, Hg2 of the respective regions of interest generated by the representative-value calculating unit 334. For example, the determining unit 337 obtains the maximal value and the minimum value of the histograms Hg3, Hg4 and compares the maximal value of one of them and the minimum value of the other one, thereby determining whether the histograms are overlapped.

When the determining unit 337 determines that there is an overlapped area in the histograms, the threshold setting unit 335 sets two thresholds in the same manner as in the above-described second embodiment (see FIG. 13). Conversely, when the determining unit 337 determines that there is no overlapped area in the histograms and the two histograms Hg3, Hg4 are separate from each other, the threshold setting unit 335 sets a single threshold in the same manner as in the above-described first embodiment (see FIG. 15). In the case illustrated in FIG. 15, the threshold setting unit 335 selects a smaller representative value among the two representative values and sets the maximal value in the region of interest as a threshold (a threshold T₁₂).

The display-specification setting unit 336 sets the display specification of the target feature value to be displayed on the display device 4 based on one or two thresholds (the first and the second thresholds) set by the threshold setting unit 335. When a single threshold is set, the display-specification setting unit 336 sets a color bar CB₄ in which color phases are changed at the threshold T₁₂ as a boundary. Specifically, in the color bar CB₄ with the threshold T₁₂ as a boundary, the side of small feature value is set in red, and the side of large one is set in blue. Furthermore, in the color bar CB₄ illustrated in FIG. 15, the red-colored area is illustrated in white, and the blue-colored area is illustrated by hatching. Conversely, when two thresholds are set, the display-specification setting unit 336 sets a color bar CB₃ in which red is set for less than the first threshold, blue is set for more than the second threshold, and color phases are gradually changed in the interval between the first and the second thresholds, in the same manner as in the second embodiment.

According to the third embodiment, the settings for the color condition (color bar) are changed depending on whether the histograms are overlapped; thus, on an ultrasound image where colors are set in accordance with the distribution of feature value, feature value on different tissue in a different region of interest is distinguishable, and, for example, even when there is a transition area of a tissue characteristic, a color pattern is recognizable.

### Fourth Embodiment

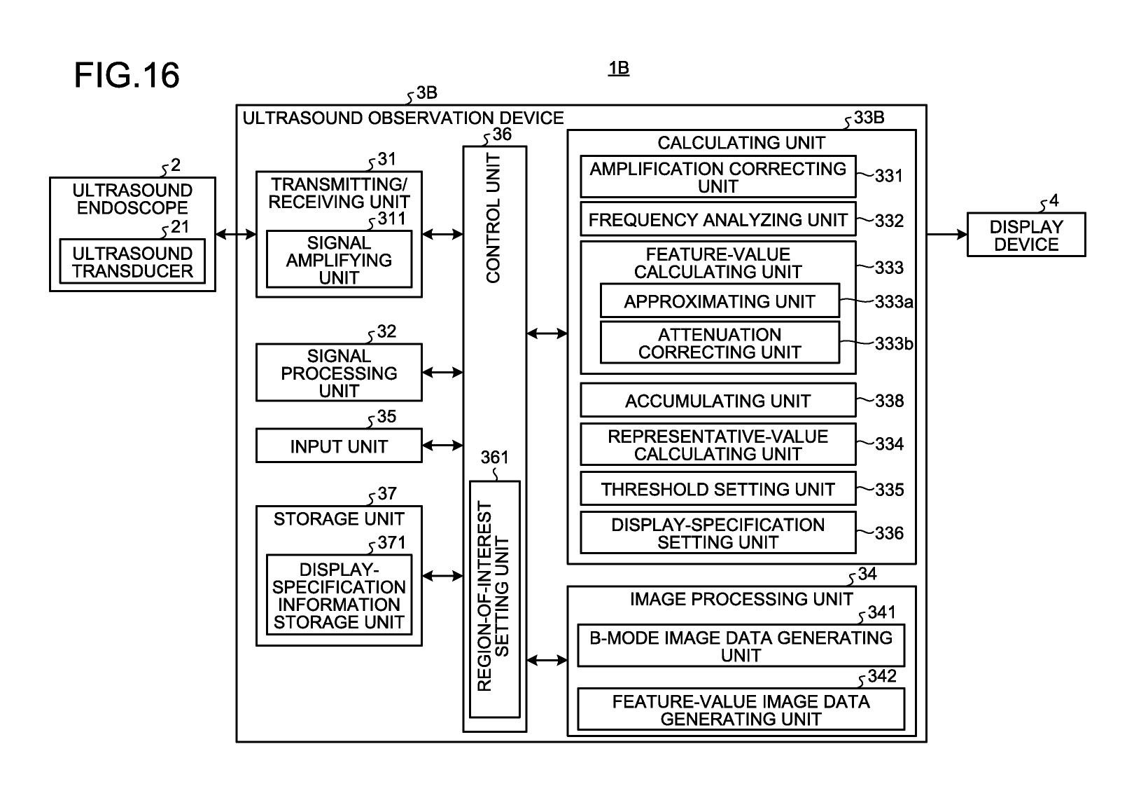

FIG. 16 is a block diagram that illustrates a configuration of an ultrasound observation system 1B including an ultrasound observation device 3B according to a fourth embodiment. The ultrasound observation system 1B illustrated in the drawing includes the ultrasound observation device 3B instead of the ultrasound observation device 3 in the ultrasound observation system 1 according to the above-described first embodiment. According to the fourth embodiment, histograms generated in accordance with sequentially acquired ultrasound signals are accumulated, a representative value is calculated by using the accumulated histograms, and a threshold and a display specification are set. A calculating unit 33B in the ultrasound observation device 3B includes an accumulating unit 338 in addition to the configuration of the above-described calculating unit 33. The other configuration is the same as that of the ultrasound observation system 1 according to the first and the second embodiments.

In the same manner as the above-described first and second embodiments, the representative-value calculating unit 334 generates the histograms Hg3, Hg4 with regard to the two set regions of interest. The representative-value calculating unit 334 sequentially stores the generated histograms in the display-specification information storage unit 371.

The accumulating unit 338 adds histograms in the region of interest, which is the same set area, stored in the display-specification information storage unit 371 and generates a cumulative histogram. When a new histogram is stored in the display-specification information storage unit 371, the accumulating unit 338 adds the histogram to the cumulative histogram.

The representative-value calculating unit 334 acquires the cumulative histogram from the accumulating unit 338 and calculates a representative value of the feature value in each region of interest based on the cumulative histogram. The subsequent threshold setting process and display-specification setting process are performed in the same manner as in the above-described first to third embodiments.

According to the fourth embodiment, the accumulating unit 338 generates a cumulative histogram by accumulating histograms stored in the display-specification information storage unit 371, and the display-specification setting unit 336 sets a display specification based on the cumulative histogram, whereby the histogram may be approximated to the normal distribution. As the histogram is approximated to the normal distribution, the reliability of the calculated representative value or the set threshold may be further improved, and as a result, the reliability of a color pattern on a displayed feature-value image may be improved so that a user is allowed to conduct high-accuracy diagnosis.

### Fifth Embodiment

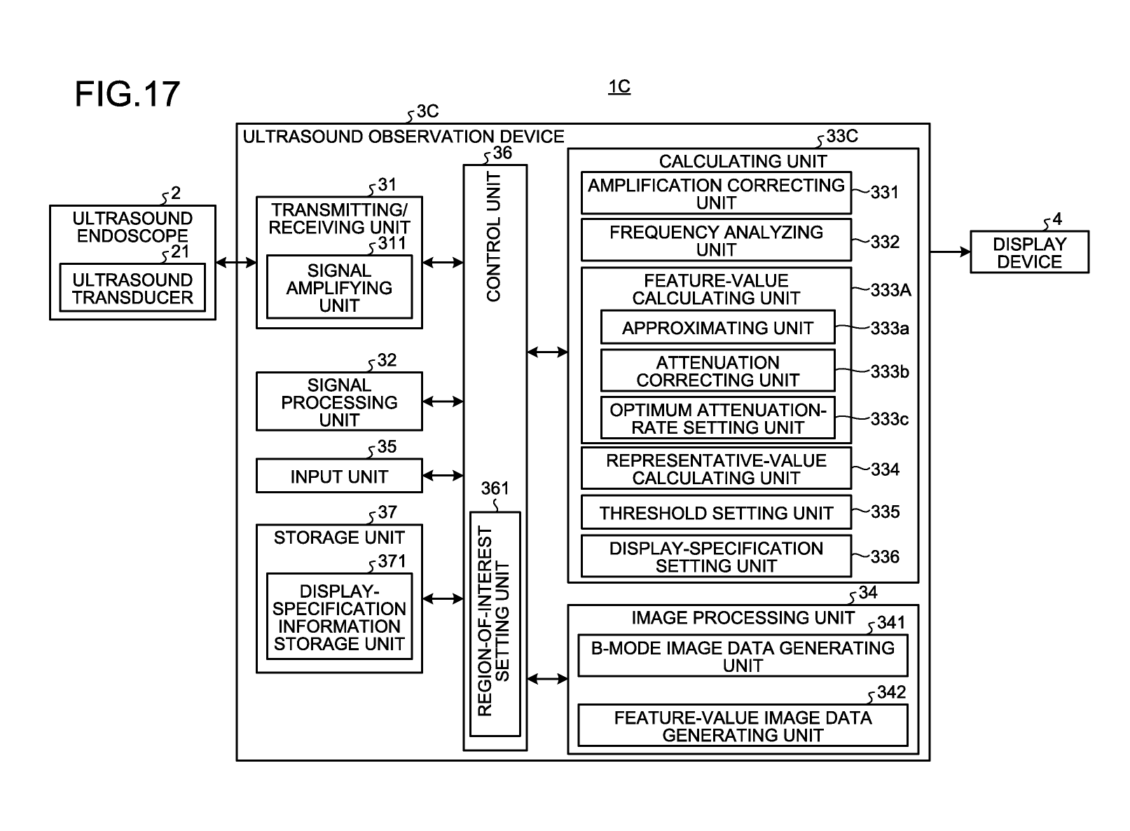

FIG. 17 is a block diagram that illustrates a configuration of an ultrasound observation system 1C including an ultrasound observation device 3C according to a fifth embodiment. The ultrasound observation system 1C illustrated in the drawing includes the ultrasound observation device 3C instead of the ultrasound observation device 3 in the ultrasound observation system 1 according to the above-described first embodiment. According to the fifth embodiment, an optimum attenuation rate is set. A calculating unit 33C in the ultrasound observation device 3C includes a feature-value calculating unit 333A instead of the feature-value calculating unit 333 in the above-described calculating unit 33. The feature-value calculating unit 333A includes an optimum attenuation-rate setting unit 333c in addition to the approximating unit 333a and the attenuation correcting unit 333b described above. The other configuration is the same as that of the ultrasound observation system 1 according to the first and the second embodiments.

The optimum attenuation-rate setting unit 333c sets the optimum attenuation rate among multiple attenuation-rate candidate values on the basis of statistical dispersion of the feature value calculated for all the frequency spectra by the attenuation correcting unit 333b.

The optimum attenuation-rate setting unit 333c sets, as the optimum attenuation rate, the attenuation-rate candidate value with the minimum statistical dispersion of the corrected feature value calculated for each attenuation-rate candidate value with regard to all the frequency spectra by the attenuation correcting unit 333b. According to the present embodiment, variance is used as a value indicating statistical dispersion. In this case, the optimum attenuation-rate setting unit 333c sets the attenuation-rate candidate value with the minimum variance as the optimum attenuation rate. Two out of the above-described three sets of feature values a, b, c are independent. In addition, the feature value b does not depend on the attenuation rate. Therefore, when the optimum attenuation rate is set for the feature values a, c, the optimum attenuation-rate setting unit 333c only needs to calculate variance of any one of the feature values a and c.

Furthermore, it is preferable that the feature value used by the optimum attenuation-rate setting unit 333c to set the optimum attenuation rate is the same type as the feature value used by the feature-value image data generating unit 342 to generate feature-value image data. Specifically, it is more preferable that, when the feature-value image data generating unit 342 generates feature-value image data by using a slope as a feature value, the variance of the feature value a is used, and when the feature-value image data generating unit 342 generates feature-value image data by using mid-band fit as a feature value, the variance of the feature value c is used. This is because Equation (1) for giving the attenuation A(f,z) is only ideal, and in reality, the following Equation (6) is more suitable.

A(f,z)=2αzf+2α₁z (6)

The second term α₁ on the right-hand side of Equation (6) is a coefficient representing the magnitude of change in the signal intensity in proportion to the receive depth z of the ultrasound wave and is a coefficient representing a change in the signal intensity which occurs due to unevenness of the target tissue to be observed, a change in the number of channels during beam synthesis, or the like. There is the second term on the right-hand side of Equation (6), and therefore, when feature-value image data is generated by using the mid-band fit as a feature value, the optimum attenuation rate is set by using the variance of the feature value c so that attenuation may be corrected more accurately (see Equation (4)). Conversely, when feature-value image data is generated by using the slope that is a coefficient proportional to the frequency f, the optimum attenuation rate is set by using the variance of the feature value a so that attenuation may be accurately corrected by removing effects of the second term on the right-hand side. For example, when the unit of the attenuation rate α is dB/cm/MHz, the unit of the coefficient α₁ is dB/cm.

Here, an explanation is given of the reason why the optimum attenuation rate is settable based on statistical dispersion. When the optimum attenuation rate is used for the observation target, it is considered that, regardless of the distance between the observation target and the ultrasound transducer 21, the feature value is converged into the value unique to the observation target and there is a little statistical dispersion. Conversely, when the attenuation-rate candidate value not suitable for the observation target is used as the optimum attenuation rate, it is considered that the feature value is deviated in accordance with the distance from the ultrasound transducer 21 due to excessive or insufficient attenuation correction and there is a larger statistical dispersion in the feature value. Therefore, it can be said that the attenuation-rate candidate value with the minimum statistical dispersion is the optimum attenuation rate for the observation target.

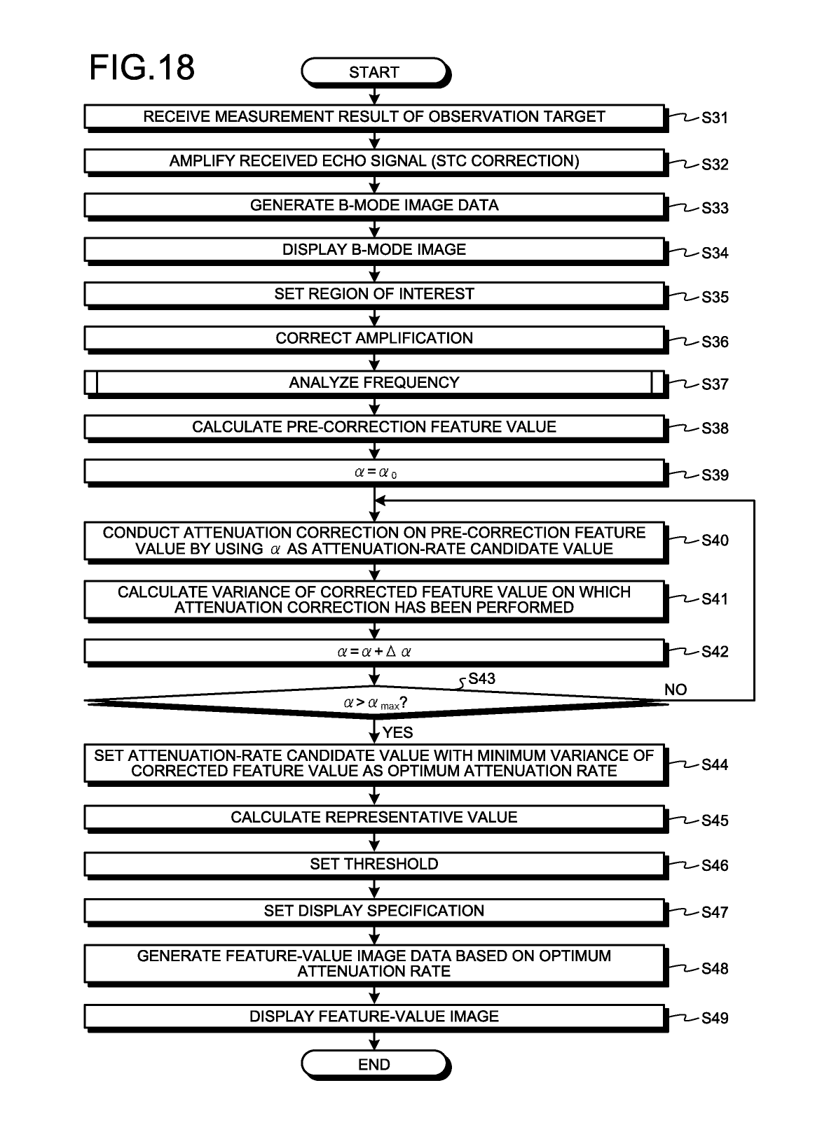

FIG. 18 is a flowchart that illustrates the outline of a process performed by the ultrasound observation device 3C. In the same manner as at Steps S1 to S8 of the flowchart illustrated in FIG. 8, the ultrasound observation device 3C calculates a pre-correction feature value (Steps S31 to S38).

Then, the optimum attenuation-rate setting unit 333c sets the value of the attenuation-rate candidate value α, which is applied when attenuation correction described later is conducted, to a predetermined default value α₀ (Step S39). The value of the default value α₀ may be previously stored in the storage unit 37 so that the optimum attenuation-rate setting unit 333c refers to the storage unit 37.

Then, the attenuation correcting unit 333b conducts attenuation correction on the pre-correction feature value, approximated by the approximating unit 333a with regard to each frequency spectrum, by using α as the attenuation-rate candidate value, to calculate a corrected feature value and stores it together with the attenuation-rate candidate value α in the display-specification information storage unit 371 (Step S40). For example, the straight line L₁ illustrated in FIG. 6 is an example of the straight line that is obtained when the attenuation correcting unit 333b performs an attenuation correction process.

At Step S40, the attenuation correcting unit 333b executes calculation by substituting the data position Z=(fₛₚ/2vₛ)Dn, which is obtained by using the data array of the sound ray of the ultrasound signal, into the receive depth z in the above-described Equations (2), (4).

The optimum attenuation-rate setting unit 333c calculates the variance of the feature value that is representative of multiple sets of feature values obtained when the attenuation correcting unit 333b conducts attenuation correction on each frequency spectrum and stores it in relation to the attenuation-rate candidate value α in the storage unit 37 (Step S41). When the feature value is the slope a and the mid-band fit c, the optimum attenuation-rate setting unit 333c calculates, for example, the variance of the feature value c. At Step S41, it is preferable that, when the feature-value image data generating unit 342 generates feature-value image data by using the slope, the optimum attenuation-rate setting unit 333c uses the variance of the feature value α and, when generating feature-value image data by using the mid-band fit, uses the variance of the feature value c.

Then, the optimum attenuation-rate setting unit 333c increases the value of the attenuation-rate candidate value α by Δα (Step S42) and compares the increased attenuation-rate candidate value α with the predetermined maximal value αₘₐₓ in magnitude (Step S43). As a result of the comparison at Step S43, when the attenuation-rate candidate value α is larger than the maximal value αₘₐₓ (Step S43: Yes), the ultrasound observation device 3C proceeds to Step S44. Conversely, as a result of the comparison at Step S43, when the attenuation-rate candidate value α is equal to or less than the maximal value αₘₐₓ (Step S43: No), the ultrasound observation device 3C returns to Step S40. In this manner, the optimum attenuation-rate setting unit 333c sets the optimum attenuation rate among the attenuation-rate candidate values within a predetermined range.

At Step S44, the optimum attenuation-rate setting unit 333c refers to the variance of each attenuation-rate candidate value stored in the display-specification information storage unit 371 and sets the attenuation-rate candidate value with the minimum variance as the optimum attenuation rate (Step S44).

Furthermore, it is also possible that, before the optimum attenuation-rate setting unit 333c sets the optimum attenuation rate, the approximating unit 333a executes regression analysis to calculate the curved line interpolating the value of the variance S(α) with regard to the attenuation-rate candidate value α and then calculates the minimum value S(α)′ₘᵢₙ in 0 (dB/cm/MHz)≤α≤1.0 (dB/cm/MHz) with regard to the curved line, thereby setting the value α′ of the attenuation-rate candidate value as the optimum attenuation rate.

Then, for each pixel of the B-mode image data generated by the B-mode image data generating unit 341, the display specification of the feature value that corresponds to the optimum attenuation rate set at Step S44 is set (Step S45 to Step S47: display-specification setting step). Step S45 to Step S47 are the same as the above-described Step S10 to Step S12.

The feature-value image data generating unit 342 superimposes visual information (e.g., color phase) based on the display specification set at Step S47 on each pixel of the B-mode image data generated by the B-mode image data generating unit 341 in association with the corrected feature value based on the optimum attenuation rate set at Step S44 and generates feature-value image data by adding information on the optimum attenuation rate (Step S48: feature-value image data generation step).

Then, the display device 4 displays the feature-value image that corresponds to the feature-value image data generated by the feature-value image data generating unit 342 under the control of the control unit 36 (Step S49). Here, the attenuation rate set as the optimum attenuation rate or the feature value that has been attenuated at the attenuation rate may be displayed.

According to the fifth embodiment, the optimum attenuation rate for the observation target is set among multiple attenuation-rate candidate values for giving different attenuation characteristics when ultrasound waves are propagated through the observation target, and attenuation correction is conducted by using the optimum attenuation rate to calculate a feature value on each of frequency spectra, whereby attenuation characteristics of ultrasound waves suitable for the observation target may be obtained by simple calculation, and observations using the attenuation characteristics may be conducted.

Furthermore, according to the fifth embodiment, the optimum attenuation rate is set based on statistical dispersion of a feature value that is obtained by conducting attenuation correction on each frequency spectrum, whereby the amount of calculation may be reduced as compared with a known technique of executing fitting with multiple attenuation models.

Furthermore, according to the fifth embodiment, for example, the optimum attenuation-rate setting unit 333c may calculate the optimum attenuation-rate equivalent value, which is equivalent to the optimum attenuation rate, in all the frames of an ultrasound image and set, as the optimum attenuation rate, the average value, the middle value, or the mode value of a predetermined number of optimum attenuation-rate equivalent values including the optimum attenuation-rate equivalent value of the latest frame. In this case, the optimum attenuation rate is less changed and the value is made stable as compared with a case where the optimum attenuation rate is set in each frame.

Furthermore, according to the fifth embodiment, the optimum attenuation-rate setting unit 333c may set the optimum attenuation rate in a predetermined frame interval of an ultrasound image. This allows the amount of calculation to be significantly reduced. In this case, until the optimum attenuation rate is subsequently set, the value of the latest optimum attenuation rate that has been set may be used.

Furthermore, according to the fifth embodiment, the target region for which statistical dispersion is calculated may be each sound ray or a region with more than a predetermined value of the receive depth. A configuration may be such that the input unit 35 is capable of receiving the setting of the above region.

Furthermore, according to the fifth embodiment, the optimum attenuation-rate setting unit 333c may set the optimum attenuation rate individually inside the set region of interest and outside the region of interest.

Furthermore, according to the fifth embodiment, a configuration may be such that the input unit 35 is capable of receiving input of a change in the setting of the default value α₀ of the attenuation-rate candidate value.

Furthermore, according to the fifth embodiment, as the value for giving statistical dispersion, it is possible to use, for example, any of the standard deviation, a difference between the maximal value and the minimum value of a feature value in the data set, and the half-value width of the distribution of a feature value. Furthermore, it is also considered that the reciprocal of variance is used as the value for giving statistical dispersion; in this case, it is obvious that the attenuation-rate candidate value with the maximum value thereof is the optimum attenuation rate.

Furthermore, according to the fifth embodiment, the optimum attenuation-rate setting unit 333c may calculate statistical dispersion of each of multiple types of corrected feature value and set the attenuation-rate candidate value with the minimum statistical dispersion as the optimum attenuation rate.

Furthermore, according to the fifth embodiment, it is also possible that the attenuation correcting unit 333b conducts attenuation correction on a frequency spectrum by using multiple attenuation-rate candidate values and then the approximating unit 333a executes regression analysis on each frequency spectrum, on which attenuation correction has been performed, to calculate a feature value.

Furthermore, according to the fifth embodiment, a feature value may be calculated in any shape, e.g., the shape formed by a command point that is input by a user via the input unit 35, other than the set region of interest.

Furthermore, in explanation according to the fifth embodiment, the optimum attenuation rate is set for each frame; however, the attenuation-rate candidate value obtained by averaging multiple frames may be used for attenuation correction. Moreover, a weight may be applied to the attenuation-rate candidate value obtained by averaging. Here, the number of frames and a weight coefficient may be set by using frame correlation in a B-mode image or may be set independently from frame correlation. For example, the number of frames used for averaging is set to 5 frames.

Furthermore, according to the fifth embodiment, it is possible to select the optimum attenuation-rate setting mode in which the optimum attenuation rate is set and attenuation correction is conducted by using the set attenuation rate and the fixed-value attenuation mode in which attenuation correction is conducted by using the previously set attenuation rate. Furthermore, it is also possible to select the fixed mode in which any of the above-described setting modes is fixed and the variable mode in which any of the above-described setting modes may be set during observation. The feature-value calculating unit 333A conducts attenuation correction in accordance with the set mode. For example, when the optimum attenuation-rate setting mode is selected while the feature-value image of the feature value obtained in the fixed-value attenuation mode is displayed, the feature-value calculating unit 333A recalculates the feature value on which attenuation correction has been performed by setting the optimum attenuation rate with regard to the feature-value image based on the echo signal, and the feature-value image data generating unit 342 generates a feature-value image by using the feature value. Here, an indication may be displayed as to whether the optimum attenuation-rate setting mode is set. For example, “ON” is displayed in green when the optimum attenuation-rate setting mode is set, and “OFF” is displayed in white when the optimum attenuation-rate setting mode is not set. Furthermore, an indication may be displayed in a different color in accordance with the calculated feature value. For example, an indication is displayed in gray when the feature value b is calculated. For example, “ON” or “OFF” is displayed immediately under attenuation correction display (the area displaying information such as the attenuation rate).

Furthermore, according to the fifth embodiment, the optimum attenuation rate may be retrieved while reducing the data amount by 8-bit quantization, or the optimum attenuation rate may be retrieved without 8-bit quantization.

Although the embodiments for carrying out the present disclosure have been explained above, the present disclosure does not need to be limited to the above-described embodiments. In explanation according to the above-described second to fifth embodiments, it is assumed that the display specification is set based on the feature values on two regions of interest in the same manner as in the first embodiment; however, this is not a limitation, and the display specification may be set based on the feature values on three regions of interest.

In the above-described first to fifth embodiments, an explanation is given of an example in which the region of interest in for example FIG. 10 is a rectangle; in addition, the one with a fan-like shape is included when the ultrasound transducer 21 is of a convex type, and the one with a circular shape is included when the ultrasound transducer 21 is of a radial type.

Furthermore, in explanation according to the above-described first to fifth embodiments, with regard to the set region of interest, the feature value is calculated, and visual information is assigned; however, with regard to the entire image, the feature value may be calculated and visual information may be assigned.

When a color bar is fixed, for example, when the color bar stored in the storage unit 37 is used, the threshold of a color phase, e.g., the lower limit value of the color bar, may be changed in accordance with a gain value of a feature-value image. Furthermore, the width of a color phase may be changed in accordance with the contrast value of a feature-value image. With regard to the threshold and the width of the above-described color phase, the reference value is set for each type of the ultrasound endoscope 2 and each feature value, and it is changed by referring to the table that is previously generated for each feature value.

Furthermore, when a color bar is fixed, it is possible to display the overview color bar representing changes in the color phase in the range of possible values of the target feature value to be displayed and the enlarged color bar in which the range from the maximal value to the minimum value of the displayed feature value is enlarged. Furthermore, the maximal value and the minimum value of the enlarged color bar are settable by a user. Furthermore, in addition to the overview color bar and the enlarged color bar, a monochrome color bar may be displayed. Furthermore, the maximal value and the minimum value of the overview color bar may be set by a user independently from a gain value, or the used color bar may be set by a user among multiple previously set color bars in which the maximal value, the minimum value, and the change form in a color phase are different. Furthermore, the noise cut level of an ultrasound image may be set by a user.

Thus, the present disclosure includes various embodiments without departing from the technical idea described in claims.

According to the present disclosure, there is an advantage such that it is possible to represent tissue characteristics in multiple regions of interest clearly and distinctively.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound observation device comprising
a processor, the processor being configured to execute:
generating an ultrasound image based on an ultrasound signal acquired by an ultrasound probe including an ultrasound transducer configured to transmit an ultrasound wave to an observation target and receive an ultrasound wave reflected by the observation target;
setting at least two regions of interest on the ultrasound image;
calculating a feature value on each of the set regions of interest based on the ultrasound signal;
calculating a representative value on each of the set regions of interest based on the calculated feature value of each of the set regions of interest;
selecting at least one representative value from the representatives values of the set regions of interest;
selecting the feature value having a predetermined relationship with the selected representatives value from the feature value used for calculating the selected representatives value;
setting the selected feature value as a threshold;
setting, as a display specification, a color pattern of the feature value to be displayed on a display based on the set threshold; and
generating feature-data image data in which the feature value, displayed together with the ultrasound image, is colored with the set display specification.

2. The ultrasound observation device according to claim 1, wherein the threshold is a value for determining a boundary between color phases that are colored on a feature-data image that corresponds to the feature-data image data.

3. The ultrasound observation device according to claim 1, wherein the processor is configured to execute setting a display specification in which a color phase is changed at the threshold as a boundary.

4. The ultrasound observation device according to claim 1, wherein the processor is configured to execute:
comparing the representative values in the respective regions of interest; and
setting, as a result of the comparison, the threshold based on the feature value on a region of interest that corresponds to a minimum representative value among the representative values.

5. The ultrasound observation device according to claim 1, wherein the processor is configured to execute
comparing the representative values in the respective regions of interest; and
setting, as a result of the comparison, the threshold based on the feature value on a region of interest that corresponds to a maximum representative value among the representative values.

6. The ultrasound observation device according to claim 1, wherein the processor is configured to execute:
setting two regions of interest;
comparing the representative values in the respective regions of interest;
setting a first threshold based on the feature value on a region of interest that corresponds to a smaller representative value among the representative values;
comparing the representative values in the respective regions of interest;
setting a second threshold based on the feature value on a region of interest that corresponds to a larger representative value among the representative values; and
setting the display specification in which the feature value equal to or more than the first threshold is colored with a color phase that corresponds to a first wavelength, the feature value equal to or less than the second threshold is colored with a color phase that corresponds to a second wavelength different from the first wavelength, and the feature value within a range between the first threshold and the second threshold is colored with a color phase that corresponds to a wavelength different from the first and the second wavelengths.

7. The ultrasound observation device according to claim 1, wherein the processor is configured to execute setting the threshold based on any of an average value, a middle value, a mode value, a standard deviation, a maximal value, and a minimum value of the feature value or a combination of two or more selected from a group thereof.

8. The ultrasound observation device according to claim 1, wherein the representative value is any of an average value, a middle value, and a mode value of the feature value.

9. The ultrasound observation device according to claim 1, wherein the processor is configured to execute:
generating, for each of the regions of interest, a histogram of a frequency of the feature value with respect to the feature value; and
accumulatively adding the histograms of regions of interest that are different from each other on the ultrasound image and that are related to each other.

10. The ultrasound observation device according to claim 1, further comprising:
a memory configured to store the display specification set by the display-specification setting unit; and
an input device configured to receive a command input for giving a command for the display specification stored in the memory, wherein
the processor is configured to execute setting the display specification in accordance with a command input received by the input device.

11. A method for operating an ultrasound observation device, the method comprising:
generating an ultrasound image based on an ultrasound signal acquired by an ultrasound probe including an ultrasound transducer configured to transmit an ultrasound wave to an observation target and receive an ultrasound wave reflected by the observation target;
setting at least two regions of interest on the ultrasound image;
calculating a feature value on each of the set regions of interest based on the ultrasound signal;
calculating a representative value on each of the set regions of interest based on the calculated feature value of each of the set regions of interest;
selecting at least one representative value from the representatives values of the set regions of interest;
selecting the feature value having a predetermined relationship with the selected representatives value from the feature value used for calculating the selected representatives value;
setting the selected feature value as a threshold;
setting, as a display specification, a color pattern of the feature value to be displayed on a display based on the set threshold; and
generating feature-value image data in which the feature value, displayed together with the ultrasound image, is colored with the set display specification.
